(19) United States
(12) Patent Application Publication
(10) Pub. No.: US 2025/0382660 A1
(43) Pub. Date: Dec. 18, 2025

(54) ENRICHMENT METHODS AND KITS

(71) Applicant: ILLUMINA, INC., San Diego, CA (US)

(72) Inventors: Gianluca Andrea Artioli, Cambridge (GB); Teodora Gal, London (GB); Angelo La Rosa, Biggleswade (GB); Nam Nguyen, Cambridge (GB); Will Tovey, Bury St. Edmunds (GB); Xavier von Hatten, Cambridge (GB)

(21) Appl. No.: 18/878,727

(22) PCT Filed: Mar. 7, 2024
(86) PCT No.: PCT/US2024/018971

§ 371 Date: Dec. 24, 2024

Related U.S. Application Data
(60) Provisional application No. 63/489,346, filed on Mar. 9, 2023.

(51) Int. Cl. C12Q 1/6813; C12Q 1/6853; C12Q 1/6876

(52) U.S. Cl. CPC C12Q 1/6813; C12Q 1/6853; C12Q 1/6876

(57) ABSTRACT

A mixture of clustered and unclustered magnetic beads are generated from magnetic beads i) functionalized with a first primer of a primer set and ii) contained in a suspension. Each clustered bead includes a first amplicon attached to the first primer and a 5′-tagged second amplicon hybridized to the first amplicon. A 5′-tag of the second amplicon is a binding pair first member. Coated non-magnetic beads (including a binding pair second member coating, and having a diameter that is at least ten times larger than each magnetic bead) are introduced into the suspension. The clustered magnetic beads bind to at least some of coated non-magnetic beads to form bead-on-bead complexes. The unclustered magnetic beads remain free in the suspension, and are separated from the suspension. The 5′-tagged second amplicon is dehybridized from the first amplicon to generate single stranded clustered magnetic beads, which are then separated from the suspension.

### CROSS-REFERENCE TO RELATED APPLICATIONS

This application claims the benefit of U.S. Provisional Application Ser. No. 63/489,346, filed Mar. 9, 2023, the content of which is incorporated by reference herein in its entirety.

### REFERENCE TO SEQUENCE LISTING

The Sequence Listing submitted herewith is hereby incorporated by reference in its entirety. The name of the file is IL1259BPCT_IP-2561-PCT_Sequence_Listing.xml, the size of the file is 7,223 bytes, and the date of creation of the file is Feb. 29, 2024.

### BACKGROUND

Various protocols in biological or chemical research involve performing a large number of controlled reactions on local support surfaces or within predefined reaction chambers. The designated reactions may then be observed or detected and subsequent analysis may help identify or reveal properties of chemicals involved in the reaction. In some examples, the controlled reactions alter charge, conductivity, or some other electrical property, and thus an electronic system may be used for detection. In other examples, the controlled reactions generate fluorescence, and thus an optical system may be used for detection.

### SUMMARY

Disclosed herein is a method for enriching a population of clustered beads in a suspension before their capture on a flow cell surface. The method enables library template seeding to take place in the suspension at a sub-Poisson ratio, which results in monoclonally clustered beads, while mitigating a loss in yield that may otherwise occur at such a low seeding ratio. To mitigate the loss in yield, the method utilizes a tagged primer to initiate clustering. As a result, tagged beads are clustered, and untagged beads remain unclustered. The tag is specifically selected to be one member of a binding pair. The other member of the binding pair is coated on another, non-magnetic bead that is introduced into the suspension post clustering. The tags enable the clustered beads to be separated from the unclustered beads.

### BRIEF DESCRIPTION OF THE FIGURES

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A through FIG. 1E together schematically depict an example of the enrichment method disclosed herein, where FIG. 1A illustrates the formation of a mixture of clustered magnetic beads and unclustered magnetic beads in a suspension of magnetic beads, FIG. 1B illustrates the formation of bead-on-bead complexes using the clustered magnetic beads in the suspension, FIG. 1C illustrates the separation of the unclustered magnetic beads from the suspension containing the bead-on-bead complexes, FIG. 1D illustrates the formation of single stranded clustered magnetic beads in the suspension, and FIG. 1E illustrates the separation of the single stranded clustered magnetic beads from the suspension;

FIG. 2 is a schematic flow diagram depicting an example of clustering that takes places during the enrichment method disclosed herein;

FIG. 3 is a top view of an example flow cell;

FIG. 4A is an enlarged, cross-sectional view, taken along the 4A-4A line of FIG. 3, depicting one example of a flow cell architecture including the single stranded clustered magnetic beads anchored to a lane;

FIG. 4B is an enlarged, cross-sectional view, taken along the 4B-4B line of FIG. 3, depicting another example of a flow cell architecture including the single stranded clustered magnetic beads anchored to posts;

FIG. 4C is an enlarged, cross-sectional view, taken along the 4C-4C line of FIG. 3, depicting yet another example of a flow cell architecture including the single stranded clustered magnetic beads anchored within depressions;

FIG. 4D is an enlarged, cross-sectional view depicting the flow cell architecture of FIG. 4C integrated into a complementary metal oxide semiconductor (CMOS) device;

FIG. 5 is a bar graph depicting the number of DNA strands (Y axis) per magnetic bead at different clustering times (hours, X axis);

FIG. 6 is a Scanning Electron Microscopy (SEM) image of bead-on-bead complexes generated during an example of the enrichment method disclosed herein; and

FIG. 7 is a bar graph depicting the number of DNA strands (Y axis) per quantitative polymerase chain reaction vial for a control sample and two example samples (X axis).

### DETAILED DESCRIPTION

The method disclosed herein generates an enriched population of clustered beads before they are introduced into a flow cell for capture and analysis. As will be described in more detail herein, the method increases i) monoclonality (i.e., instances where a single library template seeds and amplifies across a single bead), and ii) the overall yield of single stranded clustered beads that are captured on the flow cell surface. Both of these instances improve the sequencing metrics.

Flow cells for use with the enriched population of clustered beads are also disclosed herein. The flow cell substrate includes capture sites that can anchor the clustered beads at predetermined locations along the substrate.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

As used herein, the terms “anchored” and “attached” refer to the state of two things being joined, fastened, adhered, connected or bound to each other, either directly or indirectly. For example, a primer can be attached to a polymeric hydrogel by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions. Other examples of attachment include magnetic attachment.

A “binding pair” refers to two agents (e.g., materials, molecules, moieties) that have a strong affinity for one another and are capable of attaching to one another reversibly. In example binding pairs, the first member and the second member respectively include a NiNTA (nickel-nitrilotriacetic acid) ligand and a histidine tag, or streptavidin or avidin and biotin, complementary DNA strands that can hybridize to one another, functional groups that can form a disulfide bond, functional groups that can form an imine, etc. Any binding pair whose binding affinity can be reversed/released, e.g., via an external stimulus, such as pH, light, and/or temperature, can be used in the examples set forth herein.

A “capture site”, as used herein, refers to a portion of a flow cell substrate that has been modified magnetically to allow for anchoring of a single stranded clustered magnetic bead. In the examples set forth herein, the capture site may include a magnetic capture agent, a chemical capture agent, and/or an electrostatic capture agent.

A “chemical capture agent” is a material, molecule or moiety that is capable of anchoring to a functional agent of a single stranded clustered bead via a chemical mechanism. One example chemical capture agent includes a capture nucleic acid (e.g., a capture oligonucleotide) that is complementary to at least a portion of a target nucleic acid attached to the single stranded clustered bead. Still another example chemical capture agent includes a member of a binding pair that is capable of binding to a second member of a binding pair that is attached to the single stranded clustered bead. Yet another example of the chemical capture agent is a chemical reagent that is capable of forming a hydrogen bond or a covalent bond with the single stranded clustered bead. Covalent bonds may be formed, for example, through thiol-disulfide exchange, click chemistry, Diels-Alder, Michael additions, amine-aldehyde coupling, amine-acid chloride reactions, amine-carboxylic acid reactions, nucleophilic substitution reactions, etc. Some chemical capture agents may be light-triggered, i.e., activated to chemically bind to the functional agent of the clustered bead when exposed to light.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

As used herein, the term “electrostatic capture agent” refers to a charged material that is capable of electrostatically anchoring a charged or reversibly charged single stranded clustered bead. The amplicons of the clustered beads are negatively charged. As such, positively charged pads, e.g., made of silanes, polymers with azide functional groups, poly-lysine, polyimines (e.g., polyethyleneimine, polypropylene imine, etc.), and other positively charged materials, may be used as the electrostatic capture agent. Another example of an electrostatic capture agent is an electrode that can attract, when a proper voltage is applied, a reversibly chargeable functional group that is incorporated into the clustered bead. As examples, amines or carboxylic acids can be reversibly switched between a neutral and a charged species in response to a pH change, and the charged species can be attracted to the electrode. The amines or carboxylic acids may be functional groups of a polyacrylamide, poly(acrylic acid) copolymer, etc. that is coated on the magnetic core.

As used herein, the term “flow cell” is intended to mean a vessel having a reaction area where a reaction can be carried out, an inlet for delivering reagent(s) to a flow channel in fluid communication with the reaction area, and an outlet for removing reagent(s) from the flow channel. In some examples, the flow cell enables the detection of the reaction that occurs in the reaction area. For example, the flow cell may include one or more transparent surfaces allowing for the optical detection of arrays, optically labeled molecules, or the like within the flow channel.

As used herein, a “flow channel” or “channel” may be i) an enclosed area defined between two bonded components or ii) an open area defined in a single component, either of which can selectively receive a liquid sample. In some examples, the flow channel may be enclosed and defined between a substrate and a lid, and thus may be in fluid communication with capture sites positioned on the substrate. In other examples, the flow channel may be enclosed and defined between two substrate surfaces that are bonded together In still other examples, the flow channel may be open to the surrounding environment.

A “functional agent” is a material, molecule or moiety of the single stranded clustered bead that is capable of anchoring to a chemical capture site of a flow cell via a chemical mechanism. One example functional agent includes a target nucleic acid that is complementary to a capture nucleic acid (e.g., a capture oligonucleotide) on the flow cell. Still another example functional agent includes a member of a binding pair that is capable of binding to a second member of a binding pair that is attached to the flow cell.

“Functionalized magnetic beads” include a magnetic particle core, a polymeric hydrogel attached to the magnetic particle core, and a plurality of one type of primer attached to side chains or arms of the polymeric hydrogel. In some instances, the functionalized magnetic bead includes an additional mechanism to attach to a flow cell capture site.

As used herein, the term “interstitial region” refers to an area, e.g., of a substrate that separates capture sites. For example, an interstitial region can separate one capture site of an array from another capture site of the array. The two capture sites that are separated from each other can be discrete, i.e., lacking physical contact with each other. In some examples, the interstitial region is continuous whereas the capture sites are discrete, for example, as is the case for a plurality of depressions, each of which contains a capture site, defined in an otherwise continuous surface. Interstitial regions may have a surface material that differs from the surface material of the captures sites. For example, capture sites include a capture agent, and the interstitial regions can be free of the capture agent.

As used herein, the term “magnetic capture agent” refers to a magnetic material that is capable of magnetically anchoring the single stranded clustered magnetic bead. Example magnetic capture agents include ferromagnetic materials and ferrimagnetic materials.

As used herein, the term “mechanism” refers to a functional agent, a magnetic material or a reversibly chargeable functional group that is incorporated into the single stranded clustered bead in order to render the single stranded clustered bead capable of anchoring to a capture site in a flow cell.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of each primer in a primer set may be modified to allow a coupling reaction with a functional group of a polymer chain. The primer length can be any number of bases long and can include a variety of non-natural nucleotides. In an example, the sequencing primer is a short strand, ranging from 10 to 60 bases, or from 20 to 40 bases.

The term “primer set” refers to a pair of primers that together enable the amplification of a template nucleic acid strand (also referred to herein as a library template). Opposed ends of the template strand include adapters to hybridize to the respective primers in a set. In the example method set forth herein, one primer is attached to the magnetic beads and the other primer is introduced to the suspension of magnetic beads to initiate amplification and clustering.

The “single stranded clustered magnetic bead” is a magnetic core material that is functionalized with amplicons of a single library template, which are attached to the magnetic core through one primer of the primer set used in the generation of the amplicons.

The term “substrate” refers to a structure upon which various components of the flow cell (e.g., capture sites, etc.) may be added. The substrate may be a wafer, a panel, a rectangular sheet, a die, or any other suitable configuration. The substrate is generally rigid and is insoluble in an aqueous liquid. The substrate may be inert to chemistry used in capturing the single stranded clustered magnetic bead, in the sequencing reactions, etc. The substrate may be a single layer structure, or a multi-layered structure (e.g., including a support and a patterned resin on the support). Examples of suitable substrates will be described further herein.

The term “transparent” refers to a material, e.g., in the form of a layer, that is capable of transmitting a particular wavelength or range of wavelengths. For example, the material may be transparent to wavelength(s) that are used in a sequencing operation. Transparency may be quantified using transmittance, i.e., the ratio of light energy falling on a body to that transmitted through the body. The transmittance of a transparent layer will depend upon the thickness of the layer, the wavelength of light, and the dosage of the light to which it is exposed. In the examples disclosed herein, the transmittance of the transparent metal layer may range from 0.1 (10%) to 1 (100%). The material of the transparent metal layer may be a pure material, a material with some impurities, or a mixture of materials, as long as the resulting layer is capable of the desired transmittance.

### Enrichment Methods

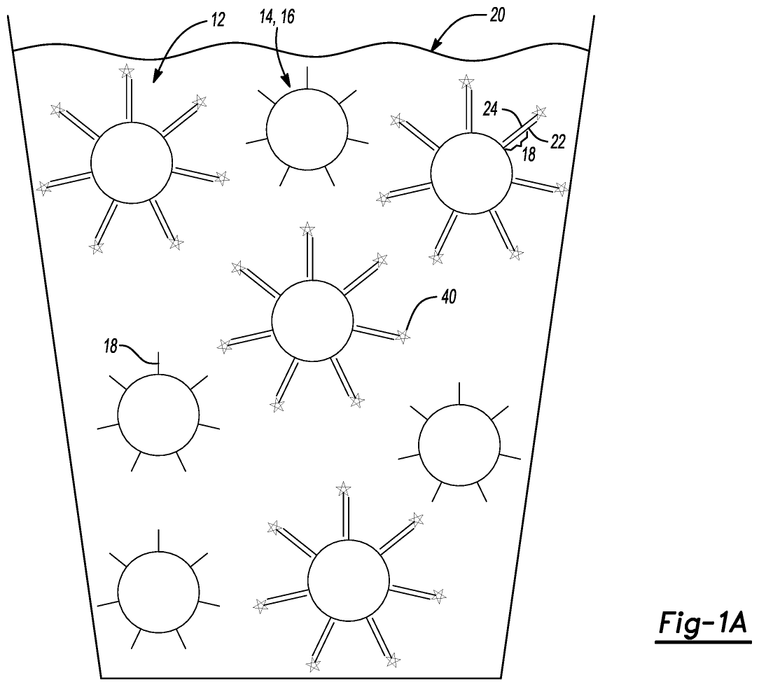

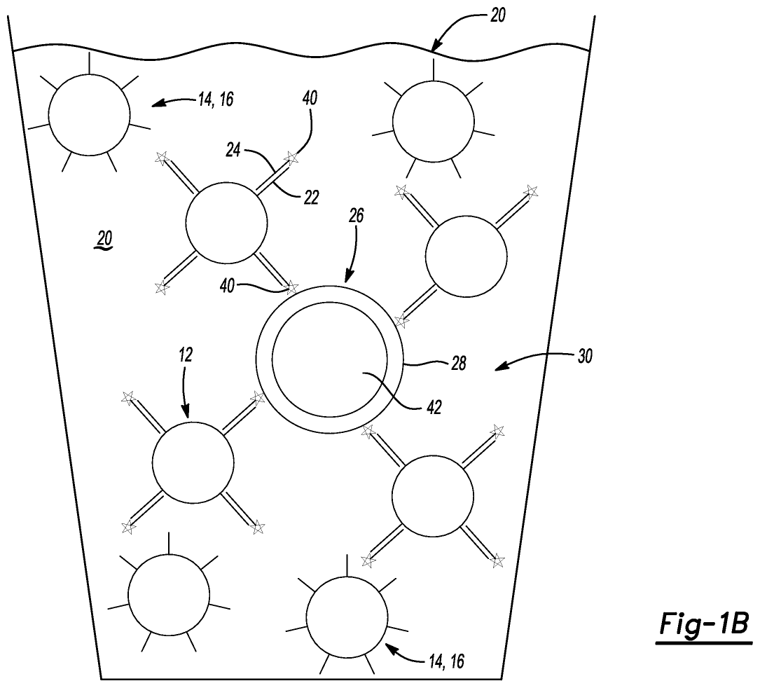

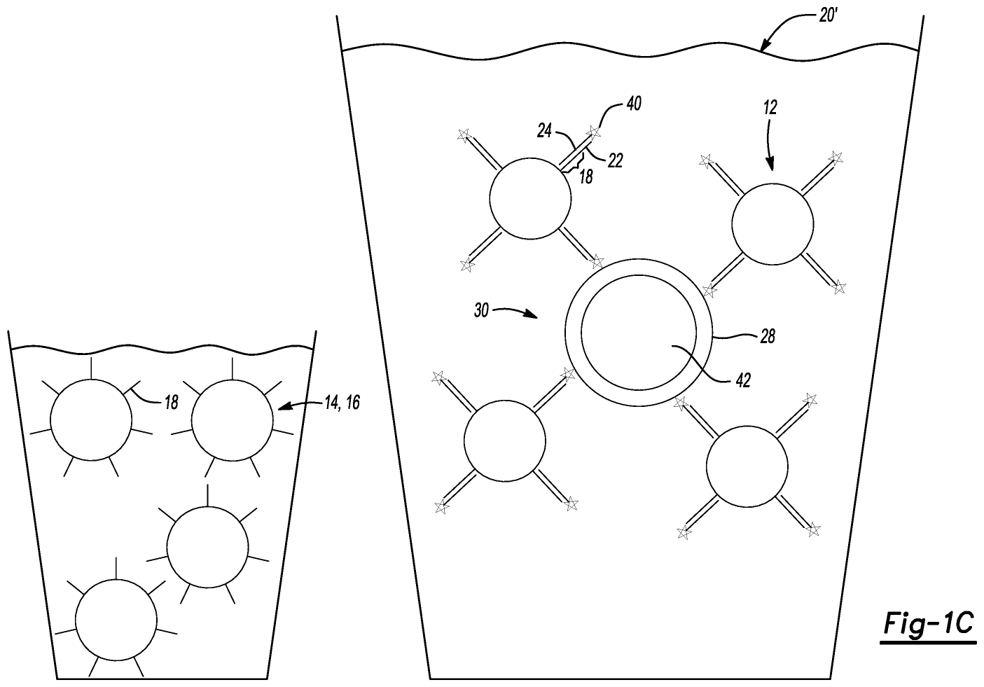

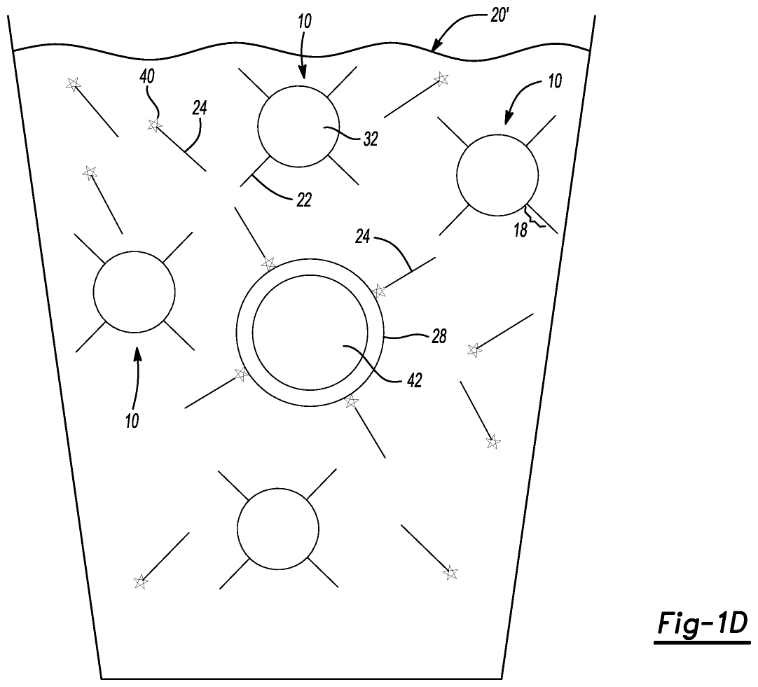

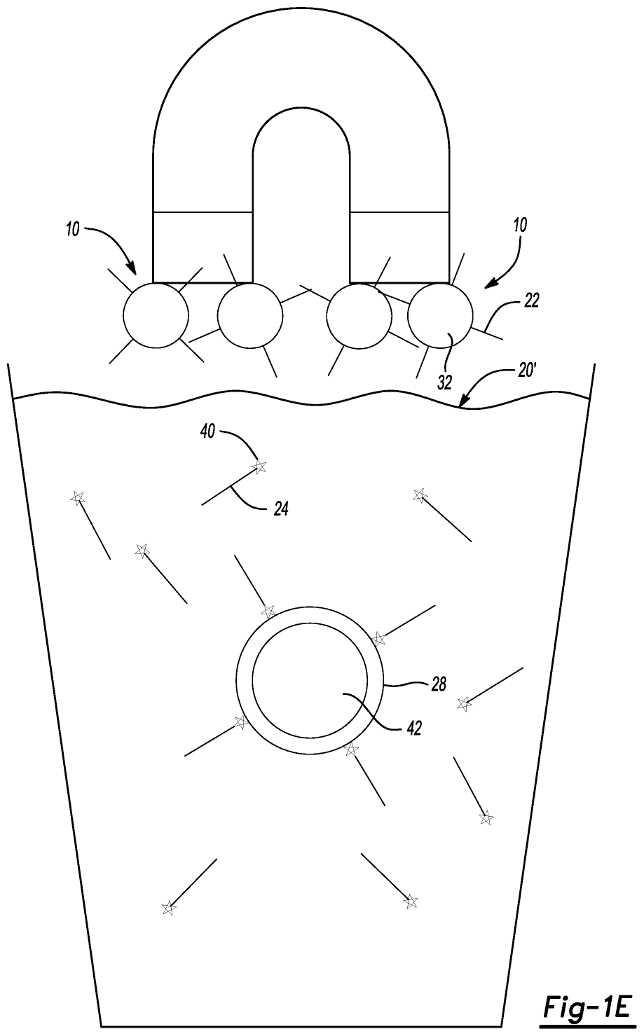

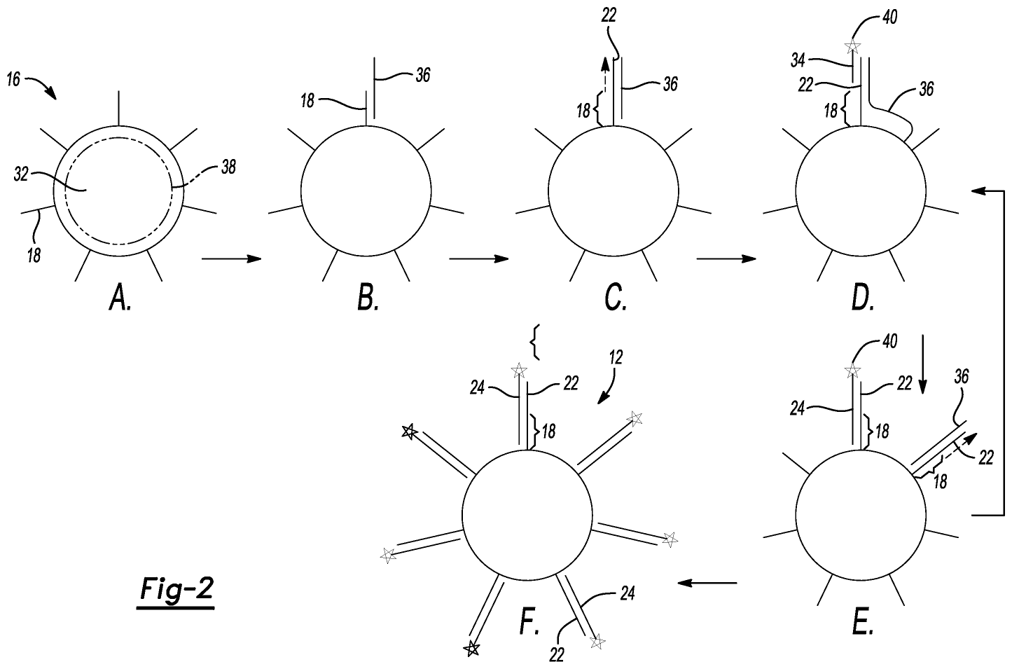

An example of the enrichment method disclosed herein is shown schematically in FIG. 1A through FIG. 1E. An example of the amplification technique used in the enrichment method is shown schematically in FIG. 2. All of these figures will be referenced throughout the description of the enrichment method.

The enrichment method generally includes generating a mixture of clustered magnetic beads 12 and unclustered magnetic beads 14 from a plurality of magnetic beads 16 i) functionalized with a first primer 18 of a primer set and ii) contained in a suspension 20, each of the clustered magnetic beads 12 including a first amplicon 22 attached to the first primer 18 and a 5′-tagged second amplicon 24 hybridized to the first amplicon 22, wherein a 5′-tag 40 of the 5′-tagged second amplicon 24 is a first member of a binding pair (see FIG. 1A and FIG. 2); introducing a plurality of coated non-magnetic beads 26 to the suspension 20, each of the plurality of coated non-magnetic beads 26 including a coating 28 of a second member of the binding pair and having a diameter that is at least ten times larger than each of the plurality of magnetic beads 16, whereby the clustered magnetic beads 12 bind to at least some of the plurality of coated non-magnetic beads 26 to form bead-on-bead complexes 30 and the unclustered magnetic beads 14 remain free in the suspension 20 (FIG. 1B); separating the unclustered magnetic beads 14 from the suspension 20 containing the bead-on-bead complexes 30 (FIG. 1C); dehybridizing the 5′-tagged second amplicon 24 from the first amplicon 22, thereby generating single stranded clustered magnetic beads 10 (FIG. 1D); and separating the single stranded clustered magnetic beads 10 from the suspension 20 (FIG. 1E).

At the outset of the enrichment method, a plurality of the magnetic beads 16 that are functionalized with the first primer 18 of a primer set are introduced into a carrier fluid to generate the suspension 20. The magnetic beads 16 are shown at “A” in FIG. 2, and will now be described.

Because the magnetic beads 16 are functionalized with the first primer 18 they are referred to herein as “functionalized magnetic beads.” The functionalized magnetic beads 16 include a magnetic core 32 and a plurality of the first primers 18 attached to the magnetic core 32. An example is depicted in FIG. 2.

The magnetic core 32 may be made of any suitable magnetic material, such as nickel, iron, cobalt, ferrites, magnetite, maghemite, or other ferromagnetic materials (e.g., particle containing Fe₃O₄ nanocomposites dispersed in a polystyrene). In an example, the magnetic core 32 is an iron oxide containing a mixture of Fe²⁺ and Fe³⁺ at an Fe²⁺:Fe³⁺ ratio ranging from about 0.5:1 to about 4:1. The magnetic core 32 that is selected should not undergo non-specific binding with the sequencing reagents or with the non-magnetic core 42.

In an example, the magnetic core 32 is a spherical nanoparticle. In another example, the magnetic core 32 is a non-spherical nanoparticle, such as a cube, a triangular prism, a rod, a platelet, a tube, etc. In still another example, the magnetic core 32 is an irregularly shaped nanoparticle. The magnetic core 32 may also be a solid structure or a hollow structure.

The dimensions of the magnetic core 32 may vary depending upon its shape. In the examples disclosed herein, the largest dimension (e.g., diameter, length, median, etc.) of the magnetic core 32 is on the nanoscale, and thus ranges from about 1 nm to less than 1000 nm. In some examples, the magnetic core 32 is a nanoparticle having a diameter of greater than or equal to 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, or greater than or equal to 100 nm. In one example, the magnetic core 32 is about 250 nm.

The magnetic material of the core 32 may be used as the mechanism for attachment to the flow cell capture site when the flow cell capture site is made of a magnetic capture agent.

The magnetic core 32 is functionalized with the first primer 18 of a primer set. As will be described in more detailed below, a second primer 34 of the primer set is introduced after a library template 36 is seeded to initiate strand invasion amplification.

Examples of suitable primers 18, 34 include P5 and P7 primers, or any combination of the PA primers, the PB primers, the PC primers, and the PD primers set forth herein. As examples, the primer set may include any two PA, PB, PC, and PD primers, or any combination of one PA primer and one PB, PC, or PD primer, or any combination of one PB primer and one PC or PD primer, or any combination of one PC primer and one PD primer. Examples of P5 and P7 primers are used on the surface of commercial flow cells sold by Illumina Inc. for sequencing, for example, on HiSeq™, HiSeqX™, MiSeq™, MiSeqDX™, MiNISeq™, NextSeq™, NextSeqDX™, NovaSeq™, iSEQ™, Genome Analyzer™, and other instrument platforms. Each of these primers 18, 34 has a universal sequence for seeding and/or amplification purposes.

The P5 primer is:

P5: 5′ → 3′
(SEQ. ID. NO. 1)
AATGATACGGCGACCACCGAGACTACAC

The P7 primer is:

P7 #1: 5′ → 3′
(SEQ. ID. NO. 2)
CAAGCAGAAGACGGCATACGAAT

The other primers (PA-PD) mentioned above include:

PA 5′ → 3′
(SEQ. ID. NO. 3)
GCTGGCACGTCCGAACGCTTCGTTAATCCGTTGAG
PB 5′ → 3′
(SEQ. ID. NO. 4)
CGTCGTCTGCCATGGCGCTTCGGTGGATATGAACT
PC 5′ → 3′
(SEQ. ID. NO. 5)
ACGGCCGCTAATATCAACGCGTCGAATCCGCAACT
PD 5′ → 3′
(SEQ. ID. NO. 6)
GCCGCGTTACGTTAGCCGGACTATTCGATGCAGC

Truncated versions of any of the primer sequences may be used, which include from 10 nucleotides to 20 nucleotides of the given sequences. The truncated versions may be shortened at either the 3′ end or the 5′ end, or at both the 3′ and 5′ ends. An example of the truncated version of P5 is:

Truncated P5: 5′ → 3′
(SEQ. ID. NO. 7)
TACGGCGACCACC

Each of the primers 18, 34 disclosed herein may also include a polyT sequence near the 5′ end of the primer sequence. In some examples, the polyT region includes from 2 T bases to 20 T bases. As specific examples, the polyT region may include 3, 4, 5, 6, 7, or 10 T bases.

The terminus at the 5′ end of the first primer 18 may be a linker that is capable of attaching to either surface functional groups of a polymeric hydrogel 38 coated on the magnetic core 32 or surface functional groups of the magnetic core 32. In either instance, the linker may be capable of single point covalent attachment. The attachment will depend, in part, on the functional groups of the hydrogel 38 or the magnetic core 32. Examples of terminated primers that may be used include an alkyne terminated primer, a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, a phosphoramidite terminated primer, and a triazolinedione terminated primer. In some specific examples, a succinimidyl (NHS) ester terminated primer may be reacted with an amine of the hydrogel 38 or the magnetic core 32, an aldehyde terminated primer may be reacted with a hydrazine of the hydrogel 38 or the magnetic core 32, or an alkyne terminated primer may be reacted with an azide of the hydrogel 38 or the magnetic core 32, or an azide terminated primer may be reacted with an alkyne or DBCO (dibenzocyclooctyne) of the hydrogel 38 or the magnetic core 32, or an amino terminated primer may be reacted with an activated carboxylate group or NHS ester of the hydrogel 38 or the magnetic core 32, or a thiol terminated primer may be reacted with an alkylating reactant (e.g., iodoacetamine or maleimide) of the hydrogel 38 or the magnetic core 32, or a phosphoramidite terminated primer may be reacted with a thioether of the hydrogel 38 or the magnetic core 32. While several examples have been provided, it is to be understood that any functional group that can be attached to the primer 18 and that can attach to a functional group of the hydrogel 38 or the magnetic core 32 may be used.

The terminus at the 5′ end of the second primer 34 is a 5′-tag 40 that is capable of binding to the coating 28 of the coated non-magnetic beads 26. Thus, the 5′-tag 40 and the coating 28 make up a binding pair. In one example, the 5′-tag 40 is biotin and the coating 28 is streptavidin.

As mentioned, the magnetic core 32 may be coated with a polymeric hydrogel 38 that includes a functional group to attach the first primer 18.

Some examples of the polymeric hydrogel 38 may at least partially encapsulate the magnetic core 32 without being covalently bonded thereto. For example, non-covalent binding could attach the polymeric hydrogel 38 to the magnetic core 32. As examples, the polymeric hydrogel 38 can attach to the magnetic core 32 through hydrogen bonding to silanols, or through physisorption via van der Waals, or through electrostatic interaction, or by affinity (lipophilic/hydrophilic).

Other examples of the polymeric hydrogel 38 may be covalently attached to the magnetic core 32, and thus the magnetic core 32 may include or be functionalized with anchoring surface groups to covalently attach to the polymeric hydrogel 38. As examples, the magnetic core 32 may be functionalized with an alkyne (e.g., dibenzocyclooctyne), and the polymeric hydrogel 38 may include an azide that can attach to the alkyne; or the magnetic core 32 may be functionalized with an azide, and the polymeric hydrogel 38 may include a dialkyne that can attached to the azide. Other covalent linkages between the polymeric hydrogel 38 and the magnetic core 32 are also possible, including those obtained through nucleophilic substitution reactions (e.g., between a nucleophilic group and a nucleofuge group). Some specific examples include those involving an aldehyde and a hydrazine, or an amine and an activated carboxylate (e.g., N-hydroxysuccinimide ester), or a thiol and an alkylating reagent, or a phosphoramidite and a thioether.

In the examples disclosed herein, the polymeric hydrogel 38 is a copolymer including at least one acrylamide monomer unit, and is a linear polymeric hydrogel or a branched polymeric hydrogel.

The linear or branched polymeric hydrogel 38 may include a first recurring unit of formula (I):

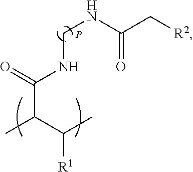

wherein:
R¹ is selected from the group consisting of —H, a halogen, an alkyl, an alkoxy, an alkenyl, an alkynyl, a cycloalkyl, an aryl, a heteroaryl, a heterocycle, and optionally substituted variants thereof; R² is selected from the group consisting of an azido, an optionally substituted amino, an optionally substituted alkenyl, an optionally substituted alkyne, a halogen, an optionally substituted hydrazone, an optionally substituted hydrazine, a carboxyl, a hydroxy, an optionally substituted tetrazole, an optionally substituted tetrazine, nitrile oxide, nitrone, sulfate, and thiol; each (CH₂)ₚ can be optionally substituted; and p is an integer from 1 to 50;
a second recurring unit of formula (II):

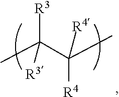

wherein: each of R³, R^{3′}, R⁴, R^{4′} is independently selected from the group consisting of —H, R⁵, —OR⁵, —C(O)OR⁵, —C(O)R⁵, —OC(O)R⁵, —C(O)NR⁶R⁷, and —NR⁶R⁷; R⁵ is selected from the group consisting of —H, —OH, an alkyl, a cycloalkyl, a hydroxyalkyl, an aryl, a heteroaryl, a heterocycle, and optionally substituted variants thereof; and each of R⁶ and R⁷ is independently selected from the group consisting of —H and an alkyl.

In an example, R¹ is —H; R² is an azido; each of R³, R⁴, and R⁴ is —H; R³ is —C(O)NR⁶R⁷, where each of R⁶ and R⁷ is —H; and p is 5. This polymeric hydrogel 14 is poly(N-(5-azidoacetamidylpentyl)acrylamide-co-acrylamide, or PAZAM. In a variation of PAZAM, R¹ is —H; R² is an azido; each of R^{3′}, R⁴, and R^{4′} is —H; R³ is —C(O)NR⁶R⁷, where each of R⁶ and R⁷ is a C1-C6 alkyl (e.g., —CH₃); and p is 5.

In some examples, R² of some of the recurring units of formula (I) is replaced with tetramethylethylenediamine (TeMED). TeMED is a reaction promoter that may be introduced during copolymerization. As a result of a side reaction, TeMED replaces some of the azide (N₃) or other R² groups. While this reaction reduces the azide (or other R² examples) content of the copolymer chains, it also introduces a branching site. The branching sites may provide a location where the copolymer chains can branch to one other.

In other examples, a third recurring unit of formula (II) may be included, with the caveat that the second and third recurring units are different. For example, in the second recurring unit each of R³, R⁴, and R⁴ is —H; R³ is —C(O)NR⁶R⁷, where each of R⁶ and R⁷ is —H, and in the third recurring unit, each of R³, R⁴, and R⁴ is —H; R³ is —C(O)NR⁶R⁷, where each of R⁶ and R⁷ is a C₁-C₆ alkyl.

The number of first recurring units (formula (I)) may be an integer ranging from 2 to 50,000, and the number of second recurring units (formula (II)) may be an integer ranging from 2 to 100,000. When the third recurring unit is included, the number of units may be an integer in the range of 1 to 100,000. It is to be understood that the incorporation of the individual units may be statistical, random, or in block, and may depend upon the method used to synthesize the polymeric hydrogel 38.

In other examples of the polymeric hydrogel 38, the first recurring unit of formula (I) may be replaced with a heterocyclic azido group of formula (III):

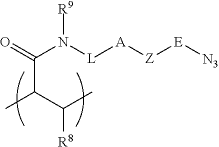

wherein R⁸ is H or a C1-C6 alkyl; R⁹ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

In one example, formula (III) is the first recurring unit and formula (II) is the second recurring unit. In another example, formula (III) is the first recurring unit, one example of formula (II) is the second recurring unit, and a different example of formula (III) is the third recurring unit.

It is to be understood that other polymeric hydrogels 38 may be used, as long as they are functionalized to graft the first primer 18 and are capable of attaching to the magnetic core 32. Some examples of suitable hydrogels 38 include functionalized polysilanes, such as norbornene silane, azido silane, alkyne functionalized silane, amine functionalized silane, maleimide silane, or any other polysilane having functional groups that can attach the first primers 18. Other examples of suitable hydrogels 38 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogels 38 include mixed copolymers of acrylamides and acrylates. A variety of polymer architectures containing acrylic monomers (e.g., acrylamides, acrylates etc.) may be utilized in the examples disclosed herein, such as highly branched polymers, including dendrimers. For example, the monomers (e.g., acrylamide, etc.) may be incorporated, either randomly or in block, into the branches (arms) of a dendrimer.

An example of the dendrimeric polymeric hydrogel 38 includes a dendritic core with recurring units of formulas (II) and (III) in the arms extending from the core. The dendritic core may have anywhere from 3 arms to 30 arms.

The dendritic core may be any multi-functional component that enables a controlled polymerization mechanism, which leads to a defined arm length in the polymer structure and an at least substantially uniform arm length between polymer structures. In an example, the arms of the dendritic core are identical to each other.

The central molecule/compound of the dendritic core may be any multi-functional molecule, such as macrocycles (e.g., cyclodextrins, porphyrins, etc.), extended pi-systems (e.g., perylenes, fullerenes, etc.), metal-ligand complexes, polymeric cores, etc. Some specific examples of the central molecule/compound of the dendritic core include a phenyl group, benzoic acid, pentraerythritol, a phosphazene group, etc.

The dendritic core includes arms that extend from the central molecule/compound. Each arm may include a group that enables the monomers of formula (II) and (III) to be incorporated. In one example, a thiocarbonylthio group is included in each arm, and thus includes a reversible addition-fragmentation chain transfer agent (a RAFT agent). In another example, the dendritic core includes an atom transfer radical polymerization (ATRP) initiator in each arm. In still another example, the dendritic core includes a nitroxide (aminooxyl) mediated polymerization (NMP) initiator in each arm.

It is to be understood that functional groups in one or more of the recurring units of the polymeric hydrogel 38 are capable of attaching the first primers 18 of the primer set. These functional groups (e.g., R², NH₂, N₃, etc.) may be located in the side chains of the linear or branched polymeric hydrogels or in the arms of the dendrimer polymeric hydrogels. These functional groups may be introduced as part of the monomer(s) used in copolymerization. To control the number of primer 18 anchorage points, the monomer bearing the functional group may be increased or decreased. These functional groups may alternatively be introduced after copolymerization.

The thickness of the polymeric hydrogel 38 on the magnetic core 32 ranges from about 10 nm to about 200 nm. The polymeric hydrogel 38 can be in a dry state or can be in a swollen state, where it uptakes liquid. The 10 nm thickness represents the polymeric hydrogel 38 in the fully dry state, and the 200 nm thickness represents the polymeric hydrogel 38 in the fully swollen state.

The weight average molecular weight of polymeric hydrogel 38 (linear or branched) ranges from about 10 kDa to about 2,000 kDa. In other examples, the weight average molecular weight ranges from about 100 kDa to about 400 kDa. Increasing the molecular weight will increase the thickness of the polymeric hydrogel 38.

The polymeric hydrogel 38 may be coated on the magnetic core 32 using any suitable deposition techniques. Examples of suitable deposition techniques include dip coating, dunk coating, spin coating, spray coating, puddle dispensing, ultrasonic spray coating, etc. In an example, the magnetic core 32 may be suspended in the polymeric hydrogel 38 and exposed to conditions (e.g., heat) that will initiate the attachment of the polymeric hydrogel 38 to the magnetic core 32.

Once the polymeric hydrogel 38 is coated on the magnetic core 32, a plurality of the first primers 18 may be grafted to the polymeric hydrogel 38. Grafting may involve dunk coating, which involves immersing the magnetic core 32 with the polymeric hydrogel 38 thereon in a primer solution or mixture, which may include the primer(s) 18, water, a buffer, and a catalyst. Other grafting techniques may involve spray coating, puddle dispensing, or another suitable method that will attach the primer(s) 18 to the polymeric hydrogel 38. With any of the grafting methods, the primers 18 react with reactive groups of the polymeric hydrogel 38. Alternatively, any of these grafting techniques may be used to attach the primers 18 directly to surface groups of the magnetic core 32 when the magnetic core 32 includes or has been functionalized with suitable functional groups for attaching the primers 18.

In other examples, a plurality of the first primers 18 may be grafted to the polymeric hydrogel 38 before it is deposited on the magnetic core 32. The magnetic core 32 may be suspended in the pre-grafted polymeric hydrogel 38 and exposed to conditions (e.g., heat) that will initiate the attachment of the pre-grafted polymeric hydrogel 38 to the magnetic core 32. In these examples, additional grafting is not performed.

Each of the functionalized magnetic beads 16 (and thus the single stranded clustered beads 10 formed therefrom) may also include or be functionalized with a mechanism that is capable of anchoring to a capture site on a flow cell substrate. The mechanism may be chemical (e.g., a functional agent), electrostatic, or magnetic.

In some examples, the mechanism is a component of the functionalized magnetic bead 16 that enables it to be anchored without further functionalization. For example, the magnetic core 32 may be the mechanism that anchors to a magnetic capture agent on the flow cell substrate. For another example, a reversibly chargeable functional group, such as an amine or a carboxylic acid, may be attached (e.g., through a thiol linkage) to the surface of the magnetic core 32 or the polymeric hydrogel 38 along with the primers 18, 34. In this example, the reversibly chargeable functional group (as the mechanism) enables the single stranded clustered beads 10 to be anchored to an electrostatic capture agent on the flow cell substrate.

In other examples, the mechanism is a functional agent that is added to the functionalized magnetic bead 16 that enables it to be anchored on the flow cell substrate. As one example, a target nucleic acid may be attached to the magnetic core 32 or the polymeric hydrogel 38 through a suitable linking group, where the target nucleic acid is complementary to a capture oligonucleotide on the flow cell substrate. As another example, a functional group for covalent attachment or a member of a binding pair may be attached to the magnetic core 32 or the polymeric hydrogel 38 through a suitable linking group. The member of the binding pair that is attached to the functionalized magnetic bead 16 for purposes of flow cell capture site attachment (i.e., is the mechanism as defined herein) may be different than the member of the binding pair that is introduced during amplification, so that the mechanism does not interfere with amplification and cluster generation.

Referring back to the enrichment method, the functionalized magnetic beads 16 are introduced into a liquid carrier to form the suspension 20. In an example, the concentration of the functionalized magnetic beads 16 ranges from about 0.05 mg/mL to about 1 mg/mL. In one specific example, the concentration of the functionalized magnetic beads 16 ranges from about 0.1 mg/mL to about 0.2 mg/mL.

The liquid carrier may be any suitable liquid in which an extension reaction, strand invasion, and amplification can take place. In an example, the liquid carrier may include water and an ionic salt buffer fluid, e.g., saline citrate, sodium citrate, sodium chloride, potassium chloride, phosphate buffered saline, etc., and other buffers, such as tris(hydroxymethyl)aminomethane (TRIS) or (4-(2-hydroxyethyl)-1-piperazineethanesulfonic acid) (HEPES). Any of the ionic salt buffers may be present at milli-molar concentrations, e.g., ranging from about 350 mM to about 800 mM. The liquid carrier may also include non-ionic surfactant(s), such as TWEEN™ polysorbates.

Generating the mixture of clustered magnetic beads 12 and unclustered magnetic beads 14 from the plurality of magnetic beads 16 in the suspension 20 involves i) introducing a plurality of library templates 36 to the suspension 20 at a magnetic bead 16:library template 36 ratio of at least 4:1, whereby some of the plurality of the library templates 36 respectively hybridize to the first primer 18 of some of the plurality of magnetic beads 16 and some other of the plurality of magnetic beads 16 remain unseeded; ii) initiating a first primer extension reaction to generate the first amplicons 22; and iii) initiating strand invasion amplification on the some of the plurality of magnetic beads 16 by introducing a 5′-tagged second primer 34 of the primer set to the plurality of magnetic beads 16.

The library templates 36 may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 18, 34 of the primer set. In some examples, the fragments from a single nucleic acid sample have the same adapters added thereto. The final library templates 36 include the DNA or cDNA fragment and adapters at both ends. The DNA or cDNA fragment represents the portion of the final library template 36 that is to be sequenced.

The plurality of library templates 36 are introduced into the suspension 20 containing the plurality of functionalized magnetic beads 16 at conditions (e.g., salt content, temperature) that enable the library templates 36 to respectively hybridize to the first primers 18 of the functionalized magnetic beads 16. In one example, the high salt content of the suspension's liquid carrier provides a suitable environment for hybridization. The temperature of the suspension 20 may be up to about 60° C. In one specific example, the seeding temperature is about 40° C.

The library templates 36 are introduced at a ratio of magnetic beads 16:library templates 36 of at least 4:1. In an example, the ratio of magnetic beads 16:library templates 36 ranges from 4:1 to 60:1. In these instances, seeding of the library templates 36 is performed at a sub-Poisson ratio. This leads to a high percentage (e.g., at least 97%) of mono-seeded magnetic beads 16 (i.e., where a single library template 36 seeds to a single bead 16 (see FIG. 2 at “B”)), and thus to a high percentage of monoclonality among the beads 16 that are seeded. However, at this/these ratio(s), several functionalized magnetic beads 16 also remain unseeded, and thus unclustered (shown at reference numeral 14 in FIG. 1A). An unclustered magnetic bead 14 does not seed a library template 36, and thus does not undergo amplification and cluster generation. Thus, the “unclustered magnetic bead” 14 is synonymous with the functionalized magnetic bead 16.

The seeding reaction may be allowed to take place for a time ranging from about 30 minutes to about 120 minutes, with or without consistent shaking.

After library template seeding, the suspension 20 may be exposed to a wash solution to remove unseeded library templates 36. The liquid carrier of the suspension 20 may also be replenished. It is to be understood that the seeded and unseeded functionalized magnetic beads 16 remain in the suspension 20 after washing. The wash solution may be an aqueous solution including any of the ionic salt buffers and surfactants set forth herein. The wash solution generally has a lower salt content than the liquid carrier of the functionalized magnetic beads 16.

A first primer extension reaction is then initiated to generate the amplicons 22, as shown at “C” in FIG. 2. To initiate the first primer extension reaction, a nucleotide mixture containing non-cleavable nucleotides and a polymerase are introduced into the suspension 20. The non-cleavable nucleotides include the following bases: adenine, cytosine, guanine and thymine. Any polymerase that can accept the non-cleavable nucleotide, and that can successfully incorporate the base of the non-cleavable nucleotide at the 3′ end of the first primer 18 may be used. Example polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 9oN (family B), and many others; polymerases from family C, such as Escherichia coli DNA Pol III, and many others, polymerases from family D, such as Pyrococcus furiosus DNA Pol II, and many others; polymerases from family X, such as DNA Pol p, DNA Pol P, DNA Pol a, and many others. The nucleotide mixture may also include a liquid carrier, such as water and/or an ionic salt buffer fluid, e.g., saline citrate at milli-molar to molar concentrations, sodium chloride, potassium chloride, phosphate buffered saline, etc., and other buffers, such as tris(hydroxymethyl)aminomethane (TRIS) or (4-(2-hydroxyethyl)-1-piperazineethanesulfonic acid) (HEPES). The liquid carrier may also include catalytic metal(s) intended for the extension reaction, such as Mg²⁺, Mn²⁺, etc. A single catalytic metal or a combination of catalytic metals may be used, and the total amount may range from about 0.01 mM to about 100 mM.

The temperature of the suspension 20 may be adjusted to initiate the template extension reaction. In an example, the temperature is about 60° C. The polymerase enables the extension of the 3′ end of the primer 18 using the library template 36 as a template for nucleotide introduction. The polymerase extension generates the amplicons 22, which are a complement of the seeded library template 36.

The extension reaction may be allowed to take place for a time ranging from about 10 minutes to about 90 minutes, with or without consistent shaking.

After the extension reaction, the suspension 20 may be exposed to a wash solution to remove unincorporated nucleotides and other components of the nucleotide mixture. The liquid carrier of the suspension 20 may also be replenished. It is to be understood that the functionalized magnetic beads 16 with the amplicons 22 attached thereto and the unseeded functionalized magnetic beads 16 remain in the suspension 20 after washing.

Strand invasion amplification may then be initiated within the suspension 20. This is illustrated in FIG. 2 at “D.” Strand invasion amplification is initiated by introducing the 5′-tagged second primer 34 to the suspension 20. The primer sequence of the 5′-tagged second primer 34 is complementary to a portion of the amplicon 22 (due to the presence of the adapter that is incorporated into the library template 36). This amplification relies on the recombinase-dependent insertion of the 5′-tagged second primer 34 into the double-stranded target nucleic acid, which is made up of the library template 36 and the first amplicon 22. The duplex regions peripheral to the insertion site dissociate, thereby enabling the 5′-tagged second primer 34 to bind. A polymerase then extends the 5′-tagged second primer 34 along the first amplicon 22 to generate the 5′-tagged second amplicon 24, which displaces the library template 36. The adapter of the displaced library template 36 that is complementary to the surface bound first primers 18 hybridizes to a nearby first primer 18, where it can be extended to generate another first amplicon 22 (as shown at “E” in FIG. 2). The strand invasion and extension reactions may be repeated, leading to exponential amplification of the library template 36 across the surface of the functionalized magnetic bead 16 (as shown at “F” in FIG. 2). This process generates the monoclonally clustered magnetic beads 12. While one example technique has been described, other non-bridging amplification techniques may be used.

The 5′-tagged second primer 34 may be introduced in a clustering mix that is free of poly(ethylene glycol) to reduce or prevent aggregation. The clustering mix includes surfactant(s) (e.g., TWEEN™ polysorbates), solvent(s) (e.g., ionic buffer(s)), polymerase(s), recombinase(s), nucleotide(s), nucleoside triphosphate(s), protein(s), other enzyme(s), reducing agent(s), and a source of magnesium.

For amplification and clustering to take place, the suspension 20 containing the clustering mix may be brought to a temperature ranging from about 37° C. to about 40° C. (e.g., about 38° C.). Amplification may be allowed to take place for a time ranging from about 30 minutes to about 90 minutes. In one example, amplification is allowed to take place for about 60 minutes (1 hour), with or without consistent shaking.

After amplification, the suspension 20 may be exposed to a wash solution to remove the clustering mix. The liquid carrier of the suspension 20 may also be replenished.

As mentioned, the method shown in FIG. 2 results in the formation of clustered magnetic beads 12 and unclustered magnetic beads 14 (i.e., those functionalized magnetic beads 16 that do not seed a library template 36). This mixture is shown in FIG. 1A, where the enrichment method begins.

Referring now to FIG. 1B, a plurality of coated non-magnetic beads 26 are added to the suspension 20. In FIG. 1B, one coated non-magnetic bead 26 is shown for simplicity of illustration.

Each coated non-magnetic bead 26 includes a non-magnetic core 42 coated with a coating 28 of the second member of the binding pair.

The non-magnetic core 42 may be any non-magnetic material. Some examples of suitable materials for the non-magnetic core 42 include polytetrafluoroethylene (PTFE), some polyacrylics, polypropylene, polyethylene, polybutylene, polyurethanes, polystyrene and other styrene copolymers, nylon (i.e., polyamide), polycaprolactone (PCL), nitrocellulose, silica (SiO₂), silica-based materials (e.g., functionalized SiO₂), and carbon. The diameter of the non-magnetic core 42 is at least ten times larger than the diameter of each of the plurality of magnetic beads 16. As such, the diameter of the non-magnetic bead 26 (including the thickness of the coating 28) is also at least ten times larger than the diameter of each of the plurality of magnetic beads 16. In an example, the diameter of the non-magnetic core 42 ranges from about 100 nm to about 10 μm. In another example, the diameter of the non-magnetic core 42 ranges from about 1 μm to about 3 μm. It is to be understood that the bigger the difference between the diameters, the easier the separation by filtration or tangential flow filtration (TFF).

As mentioned, the coating 28 of the coated non-magnetic bead 26 is the second member of the binding pair, and thus depends upon the 5′-tag 40 of the 5′-tagged second primer 34, which is the first member of the binding pair. In one example, the coated non-magnetic beads 26 are coated silica beads (i.e., the non-magnetic core 42 is silica), and the coating 28 is streptavidin.

The coated non-magnetic beads 26 may be commercially available, or may be prepared by depositing streptavidin over the non-magnetic core 42 using any of the deposition techniques set forth herein.

The plurality of coated non-magnetic beads 26 is added to the suspension 20 at a magnetic bead 16:coated non-magnetic bead 26 ratio ranging from greater than 1:1 to 300:1. This ratio may be higher if a larger non-magnetic bead 26 is used. In this ratio, the number of magnetic beads 16 reflects the approximate or actual number of magnetic beads 16 that is present at the outset of the method. The ratio may be adjusted depending upon the diameters of the beads 16, 26. It is desirable for the clustered magnetic beads 12 to attach to the coating 28 of the coated non-magnetic beads 26 without aggregating. In some instances, the ratio may be selected so that a desired number of the smaller magnetic beads 16 can substantially uniformly attach across the surface of the larger coated non-magnetic bead 26 without being overcrowded. To avoid aggregation with any size beads 16, 26, the surface of the non-magnetic bead 26 should not be covered more than 50% by the magnetic beads 16. The negative charge of the clustered beads 10 should help mitigate aggregation as well.

The plurality of coated non-magnetic beads 26 may be introduced into the suspension 20 in a buffer containing from about 0.1% active (w/v) to about 0.5% active (w/v) of a non-ionic surfactant. The salt content in the buffer can facilitate the bioconjugation of the first and second members of the binding pair, and the non-ionic surfactant can aid in reducing aggregation of the clustered magnetic beads 12. The mixture shown in FIG. 1B may be allowed to incubate at room temperature (e.g., from about 18° C. to about 22° C.) for up to 24 hours, with or without consistent shaking. Incubation may range from about 30 minutes to about 24 hours, depending upon the bead ratio (bead 16:bead 26) and the bonding chemistry that is utilized. In one example, incubation takes place for about 16 hours.

Because the clustered magnetic beads 12 include the first member of the binding pair (as the 5′-tag 40 of second amplicon 24) and coated non-magnetic beads 26 include the second member of the binding pair (as the coating 28), the clustered magnetic beads 12 bind to the surface of the coated non-magnetic beads 26 during incubation. Similarly, because the unclustered magnetic beads 14 do not include the first member of the binding pair (as no amplicons 22, 24 are generated on these beads 14, 16), the unclustered magnetic beads 14 do not bind to the surface of the coated non-magnetic beads 26, but rather remain dispersed throughout the liquid carrier of the suspension 20, as depicted in FIG. 1B. This process forms bead-on-bead complexes 30 that enable the separation of the unbound (free), unclustered magnetic beads 14 from the bound, clustered magnetic beads 12.

After the plurality of coated non-magnetic beads 26 is added to the suspension 20 and incubated, the unclustered magnetic beads 14 are separated from the suspension 20 containing the bead-on-bead complexes 30. This is depicted in FIG. 1C. In one example, the unclustered magnetic beads 14 are filtered from the suspension. This may be accomplished using a membrane filtration system, such as tangential flow filtration (TFF). This system and technique allows the smaller unclustered magnetic beads 14 to be removed and reused. Moreover, the modularity of this system and technique allows for different membrane pore sizes, thus allowing unclustered magnetic beads 14 of a variety of sizes to be removed. Other techniques that may be used include sedimentation filtration (where the bead-on-bead complexes 30 will sediment faster than the unclustered magnetic beads 14), or differential magnetic pull (where the bead-on-bead complexes 30 will be pulled at a different speed than the unclustered magnetic beads 14), or standard filtration with a size cut off just below the size of the bead-on-bead complexes 30. Regardless of the technique used, the removal of the unclustered magnetic beads 14 leaves a suspension that is enriched with clustered magnetic beads 12.

As shown in FIG. 1D, the enrichment method then involves the dehybridization of the 5′-tagged second amplicon 24 from the first amplicon 22, which generates single stranded clustered magnetic beads 10. This releases the single stranded clustered magnetic beads 10 from the bead-on-bead complexes 30 without having to disrupt the binding pair linkage (between the 5′-tag 40 and the coating 28).

In one example, the dehybridization of the 5′-tagged second amplicon 24 from the first amplicon 22 involves introducing, to the suspension 20, a basic solution that denatures the 5′-tagged second amplicon 24 from the first amplicon 22 and that dissolves at least a portion of the plurality of coated non-magnetic beads 26. As an example, the basic solution may be an aqueous solution of sodium hydroxide (NaOH). The molarity of the solution may range from about 0.01 M NaOH to about 1 M. The dissolution of at least a portion of the coated non-magnetic bead 26 helps with dehybridzation because additional smaller molecules often added to compete for rehybridization do not have to be introduced.

The suspension 20 (containing the bead-on-bead complexes 30) and the basic solution may be allowed to incubate at a temperature ranging from about 18° C. to about 37° C. for a time ranging from about 3 minutes to about 10 minutes. This process may also be performed with or without continuous shaking.

In another example, the dehybridization of the 5′-tagged second amplicon 24 from the first amplicon 22 involves introducing, to the suspension 20, formamide. 100% formamide may be used, or a diluted sample containing from 50% to less than 100% of formamide may be used.

The suspension 20 (containing the bead-on-bead complexes 30) and the formamide may be allowed to incubate at a temperature ranging from about 55° C. to about 95° C. for a time ranging from about 10 minutes to about 30 minutes. Multiple stages of dehybridization may be performed, where each ranges from about 5 minutes to about 15 minutes. This process may also be performed with or without continuous shaking.

Each of these examples separates the 5′-tagged second amplicon 24 from the first amplicon 22, which remains attached to the magnetic core 32 through the first primers 18. Thus, the clustered magnetic beads 12 are released from the coated non-magnetic beads 26, even though the corresponding 5′-tagged second amplicons 24 remain attached to the coated non-magnetic beads 26. As depicted in FIG. 1D, this process also releases the 5′-tagged second amplicons 24 that were hybridized to the first amplicons 22 but not linked to the coating 28. It is to be understood that the first primers 22 remain intact on the clustered magnetic beads 12, even when a portion of the coated non-magnetic beads 26 is dissolved.

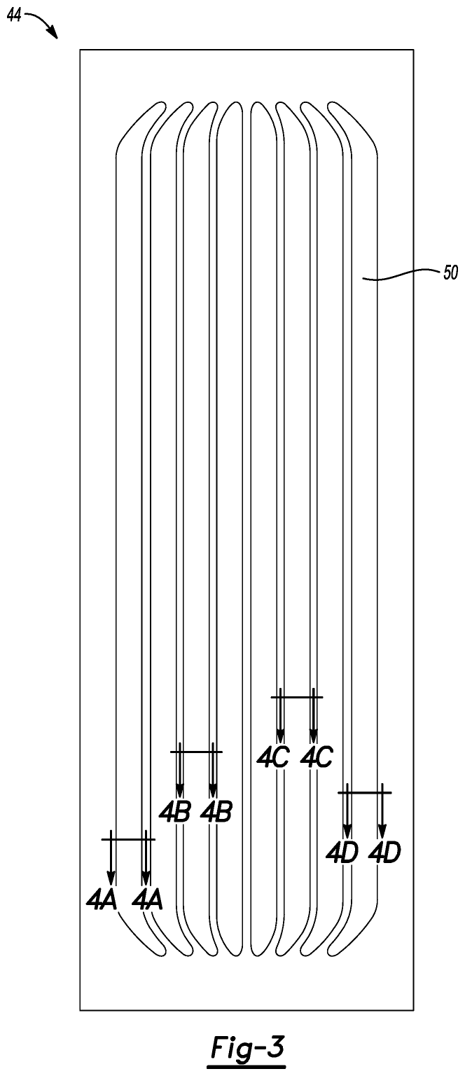

The single stranded clustered magnetic beads 10 are then separated from the suspension 20 (FIG. 1E). Because the single stranded clustered magnetic beads 10 are magnetic and the coated non-magnetic beads 26 are not magnetic, the single stranded clustered magnetic beads 10 can be removed using magnetic pull. Any suitable magnet may be used. The separated single stranded clustered magnetic beads 10 can be washed (as described herein) and then introduced into a flow cell (reference numeral 44 in FIG. 3) for a sequencing operation.

### Flow Cells

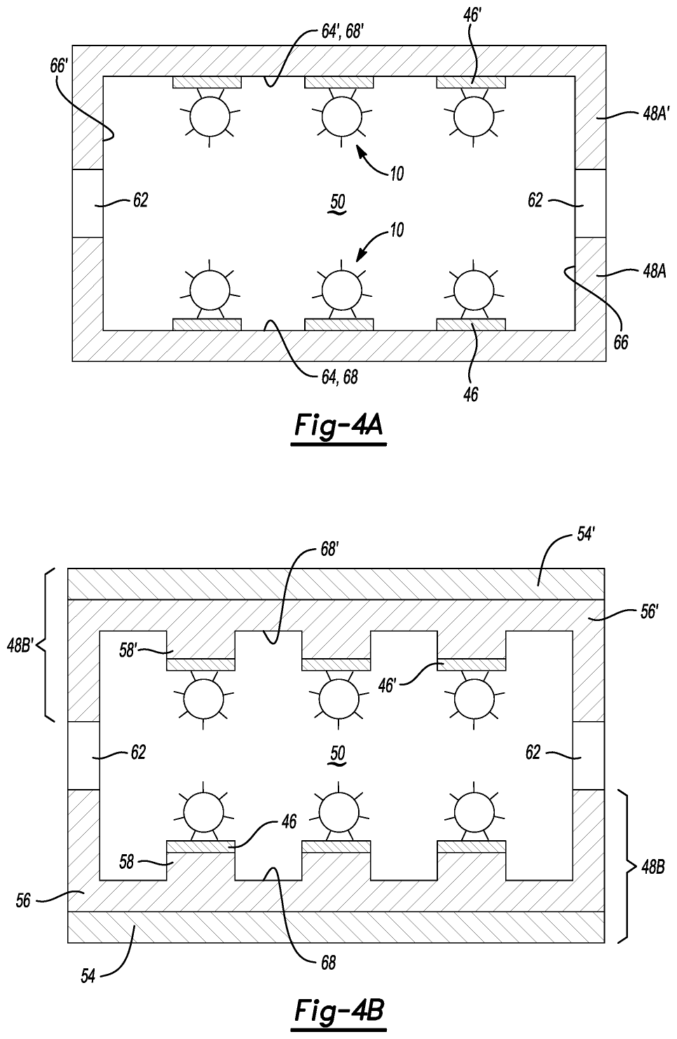

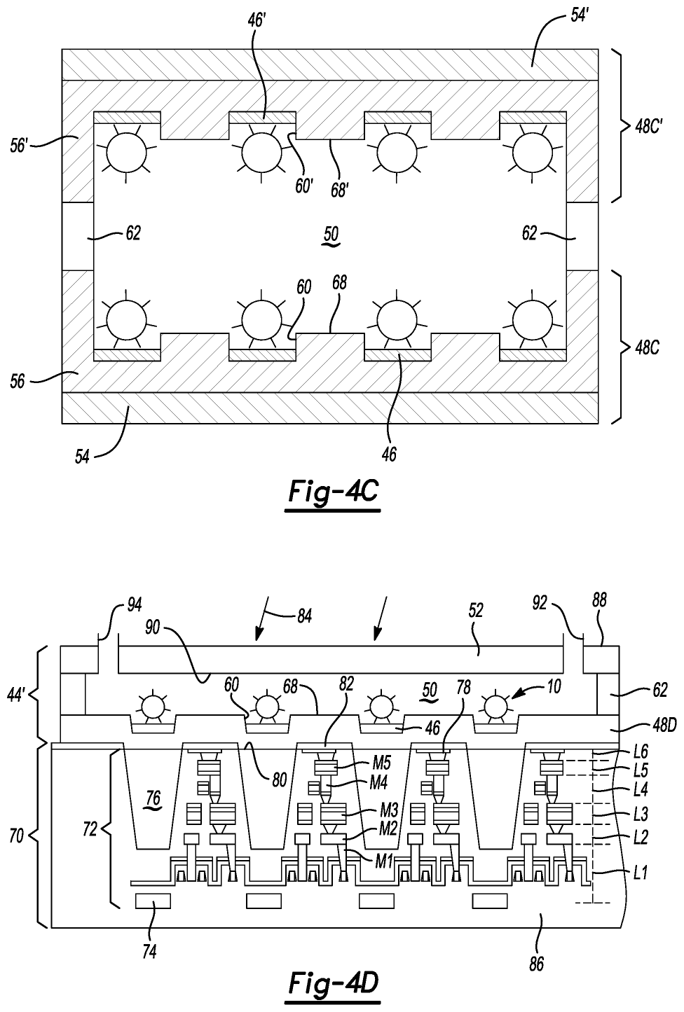

The separated single stranded clustered magnetic beads 10 may be used with any flow cell 44 (FIG. 3) that includes capture sites 46, 46′ (FIG. 4A through FIG. 4D. An example of the flow cell 44 is depicted from the top view in FIG. 3, and different examples of the flow cell architecture, including different configurations of the capture sites 46, 46′, are shown in FIG. 4A, FIG. 4B, FIG. 4C, and FIG. 4D.

A top view of an example of the flow cell 44 is shown in FIG. 3. As will be discussed in reference to FIG. 4A, FIG. 4B, and FIG. 4C, some examples of the flow cell 44 include two opposed substrates 48A, 48A′ or 48B, 48B′ or 48C, 48C′, each of which is configured with capture sites 46, 46′. In these examples, a flow channel 50 is defined between the two opposed substrates 48A, 48A′ or 48B, 48B′ or 48C, 48C′. In other examples, the flow cell 44 includes one substrate 48A or 48B or 48C configured with capture sites 46 and a lid 52 (see FIG. 4D) attached to the substrate 48A or 48B or 48C. In these examples, the flow channel 50 is defined between the substrate 48A or 48B or 48C and the lid 52.

Different substrates 48A, 48A′ or 48B, 48B′ or 48C, 48C′ are shown in FIG. 4A, FIGS. 4B, and 4C.

In the example shown in FIG. 4A, the substrates 48A, 48A′ are single layered structures. Examples of suitable single layered structures for the substrate 48A, 48A′ include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

In the examples shown in FIG. 4B and FIG. 4C, the substrates 48B, 48B′ and 48C, 48C′ are multi-layered structures. The multi-layered structures of the substrates 48B, 48B′ and 48C, 48C′ include a base support 54, 54′ and a patterned material 56, 56′ on the base support 54, 54′.

The base support 54, 54′ may be any of the examples set forth herein for the single layered structure of the substrate 48A, 48A′.

The patterned material 48A, 48A′ may be any material that is capable of being patterned with posts 58, 58′ (FIG. 4B) or depressions 60, 60′ (FIG. 4C).

In an example, the patterned material 56, 56′ may be an inorganic oxide that is selectively applied to the base support 54, 54′, e.g., via vapor deposition, aerosol printing, or inkjet printing, in the desired pattern. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

In another example, the patterned material 56, 56′ may be a resin matrix material that is applied to the base support 54, 54′ and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, printing techniques, etc. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” (commercially available under the tradename POSS® from Hybrid Platics) refers to a chemical composition that is a hybrid intermediate (e.g., RSiO_{1.5}) between that of silica (SiO₂) and silicone (R₂SiO). An example of polyhedral oligomeric silsesquioxane can be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may comprise one or more different cage or core structures as monomeric units. The average cage content can be adjusted during the synthesis, and/or controlled by purification methods, and a distribution of cage sizes of the monomeric unit(s) may be used in the examples disclosed herein.

In an example, the substrates 48A, 48A′ or 48B, 48B′ or 48C, 48C′ (whether single or multi-layered) may be round and have a diameter ranging from about 2 mm to about 300 mm, or may be a rectangular sheet or panel having its largest dimension up to about 10 feet (˜3 meters). In an example, the substrate 48A, 48A′ or 48B, 48B′ or 48C, 48C′ is a wafer having a diameter ranging from about 200 mm to about 300 mm. Wafers may subsequently be diced to form an individual flow cell substrate. In another example, the substrate 48A, 48A′ or 48B, 48B′ or 48C, 48C′ is a die having a width ranging from about 0.1 mm to about 10 mm. While example dimensions have been provided, it is to be understood that a substrate 48A, 48A′ or 48B, 48B′ or 48C, 48C′ with any suitable dimensions may be used. For another example, a panel may be used that is a rectangular support, which has a greater surface area than a 300 mm round wafer. Panels may subsequently be diced to form individual flow cells.

The flow cell 44 also includes the flow channel 50. While several flow channels 50 are shown in FIG. 3, it is to be understood that any number of flow channels 50 may be included in the flow cell 44 (e.g., a single channel 50, four channels 50, etc.). Each flow channel 50 may be isolated from each other flow channel 50 in a flow cell 44 so that fluid introduced into any particular flow channel 50 does not flow into any adjacent flow channel 50.

A portion of the flow channel 50 may be defined in the substrate 48A, 48A′ or 48B, 48B′ or 48C, 48C′ using any suitable technique that depends, in part, upon the material(s) of the substrate 48A, 48A′ or 48B, 48B′ or 48C, 48C′. In one example, a portion of the flow channel 50 is etched into a glass substrate, such as substrate 48A, 48A′. In another example, a portion of the flow channel 50 may be patterned into a resin matrix material of a multi-layered structure using photolithography, nanoimprint lithography, etc. A separate material (e.g., material 62 in FIG. 4A through FIG. 4D may be applied to the substrate 48A, 48A′ or 48B, 48B′ or 48C, 48C′ so that the separate material 62 defines at least a portion of the walls of the flow channel 50.

In an example, the flow channel 50 has a substantially rectangular configuration with rounded ends. The length and width of the flow channel 50 may be smaller, respectively, than the length and width of the substrate 48A, 48A′ or 48B, 48B′ or 48C, 48C′ so that a portion of the substrate surface surrounding the flow channel 50 is available for attachment to another substrate 48A, 48A′ or 48B, 48B′ or 48C, 48C′ or to a lid 52. In some instances, the width of each flow channel 50 can be at least about 1 mm, at least about 2.5 mm, at least about 5 mm, at least about 7 mm, at least about 10 mm, or more. In some instances, the length of each flow channel 50 can be at least about 10 mm, at least about 25 mm, at least about 50 mm, at least about 100 mm, or more. The width and/or length of each flow channel 50 can be greater than, less than or between the values specified above. In another example, the flow channel 50 is square (e.g., 10 mm×10 mm).

The depth of each flow channel 50 can be as small as a few monolayers thick, for example, when microcontact, aerosol, or inkjet printing is used to deposit the separate material 62 that defines the flow channel walls. In other examples, the depth of each flow channel 50 can be about 1 μm, about 10 μm, about 50 μm, about 100 μm, or more. In an example, the depth may range from about 10 μm to about 100 μm. In another example, the depth is about 5 μm or less. It is to be understood that the depth of each flow channel 50 can also be greater than, less than or between the values specified above. The depth of the flow channel 50 may also vary along the length and width of the flow cell 44, e.g., when posts 58, 58′ or depressions 60, 60′ are used.

In the example shown in FIG. 4A, each substrate 48A, 48A′ has a substantially flat surface 64, 64′; and the plurality of capture sites 46, 46′ are positioned in a pattern across the substantially flat surfaces 64, 64′.

The substantially flat surfaces 64, 64′ may be the bottom surface of lanes 66, 66′ that are defined in the single layer substrate 48A, 48A′. A lane 56, 56′ may also be defined in the patterned layer 64, 64′ of a multi-layered substrate 48B, 48B′, 48C, 48C′. The lanes 56, 56′ may be etched into the substrate or defined, e.g., by lithography or another suitable technique.

The plurality of capture sites 46, 46′ are positioned in a pattern across the substantially flat surface 64, 64′.

Many different patterns for the capture sites 46, 46′ may be envisaged, including regular, repeating, and non-regular patterns. In an example, the capture sites 46, 46′ are disposed in a hexagonal grid for close packing and improved density. Other layouts may include, for example, rectilinear (rectangular) layouts, triangular layouts, and so forth. In some examples, the layout or pattern can be an x-y format of capture sites 46, 46′ that are in rows and columns. In some other examples, the layout or pattern can be a repeating arrangement of capture sites 46, 46′ separated by regions of the substantially flat surface 64, 64′. In still other examples, the layout or pattern can be a random arrangement of capture sites 46, 46′. The pattern may include stripes, swirls, lines, triangles, rectangles, circles, arcs, checks, diagonals, arrows, and/or squares.

The layout or pattern of the capture sites 46, 46′ may be characterized with respect to the density of the capture sites 46, 46′ (e.g., number of capture sites 46, 46′) in a defined area. For example, the capture sites 46, 46′ may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of capture sites 46, 46′ can be between one of the lower values and one of the upper values selected from the ranges above. As examples, a high density array may be characterized as having capture sites 46, 46′ separated by less than about 100 nm, a medium density array may be characterized as having capture sites 46, 46′ separated by about 400 nm to about 1 μm, and a low density array may be characterized as having capture sites 46, 46′ separated by greater than about 1 μm. While example densities have been provided, it is to be understood that any suitable densities may be used. In some instances, it may be desirable for the spacing between capture sites 46, 46′ to be even greater than the examples listed herein.

The layout or pattern of the capture sites 46, 46′ may also or alternatively be characterized in terms of the average pitch, or the spacing from the center of one capture site 46, 46′ to the center of an adjacent capture site 46, 46′ (center-to-center spacing) or from the left edge of one capture site 46, 46′ to the right edge of an adjacent capture site 46, 46′ (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of capture sites 46, 46′ can be between one of the lower values and one of the upper values selected from the ranges above. In an example, the capture sites 46, 46′ have a pitch (center-to-center spacing) of about 1.5 μm. While example average pitch values have been provided, it is to be understood that other average pitch values may be used.

The capture sites 46, 46′ may have any suitable shape, geometry and dimensions, which may depend, at least in part, on the single stranded clustered magnetic bead 10 that is to be captured by the capture site 46, 46′.

The capture sites 46, 46′ may be magnetic capture sites. chemical capture sites, or electrostatic captures sites.

In some of the examples set forth herein, the capture sites 46, 46′ are magnetic capture sites. Magnetic capture sites include any example of the magnetic capture agent set forth herein that can be deposited on predefined locations of the substantially flat surface 64, 64′. For example, magnetic materials may be deposited using chemical vapor deposition, masking and deposition, or another suitable technique to form the capture sites 46, 46′.

In other of the examples set forth herein, the capture sites 46, 46′ are chemical capture sites. Chemical capture sites include any example of the chemical capture agent set forth herein that can be deposited on or otherwise attached to predefined locations of the substantially flat surface 64, 64′. In one example, the chemical capture agent may be deposited, e.g., using microcontact printing, aerosol printing, etc., in a desirable location on the substantially flat surface 64, 64′ to form the capture sites 46, 46′. In another example, a mask (e.g., a photoresist) may be used to define the space/location where the chemical capture agent will be deposited. The chemical capture agent may then be deposited, and the mask removed (e.g., via lift-off, dissolution, or another suitable technique). In this example, the chemical capture agent may form a monolayer or thin layer of the chemical capture agent. In still another example, a polymer grafted with capture nucleic acids may be selectively applied to the substantially flat surface 64, 64′ to form the chemical captures sites.

In still other of the examples set forth herein, the capture sites 46, 46′ are electrostatic capture sites. Electrostatic captures sites include any example of the electrostatic capture agents set forth herein that can be deposited on predefined locations of the substantially flat surface 64, 64′. For example, electrode materials may be deposited using chemical vapor deposition, masking and deposition, or another suitable technique to form the capture sites 46, 46′. When electrostatic capture sites are used, the substrate 48A, 48A′ may include additional circuitry to address the individual capture sites 46, 46′.

In the example of FIG. 4A, areas of the substantially flat surface 64, 64′ that do not contain the capture sites 46, 46′ function as interstitial regions between the capture sites 46, 46′.

In the example shown in FIG. 4B, the substrate 48B, 48B′ includes posts 58, 58′ separated by interstitial regions 68, 68′; and a capture site 46, 46′ is positioned over each of the posts 58, 58′.

Each post 58, 58′ is a three-dimensional structure that extends outward (upward) from an adjacent surface. The post 58, 58′ is thus a convex region with respect to the interstitial regions 68, 68′ that surround the posts 58, 58′. Posts 58, 58′ may be formed in or on a substrate 48B, 48B′. In FIG. 4B, the posts 58, 58′ are formed in the substrate 48B, 48B′. When the post 58, 58′ is formed “in the substrate”, it is meant that a is patterned (e.g., via etching, photolithography, imprinting, etc.) so that the resulting patterned material 56, 56′ includes posts 58, 58′ that extend above the adjacent surrounding interstitial regions 68, 68′. Alternatively, when the post 58, 58′ is formed “on the substrate”, it is meant that an additional material may be deposited on the substrate (e.g., on the single layer substrate) so that it extends above the underlying substrate.

The layout or pattern of the posts 58, 58′ may be any of the examples set forth herein for the capture sites 46, 46′. The layout or pattern of the posts 58, 58′ may be characterized with respect to the density of the posts 58, 58′ (e.g., number of posts 58, 58′) in a defined area. Any of the densities set forth for the capture sites 46, 46′ may be used for the posts 58, 58′. The layout or pattern of the posts 58, 58′ may also be characterized in terms of the average pitch, or the spacing from the center of one post 58, 58′ to the center of an adjacent post 58, 58′ (center-to-center spacing) or from the left edge of one post 58, 58′ to the right edge of an adjacent post 58, 58′ (edge-to-edge spacing). Any of the average pitches set forth for the capture sites 46, 46′ may be used for the posts 58, 58′.

While any suitable three-dimensional geometry may be used for the posts 58, 58′, a geometry with an at least substantially flat top surface may be desirable so that the capture site 46, 46′ may be formed thereon. Example post geometries include a sphere, a cylinder, a cube, polygonal prisms (e.g., rectangular prisms, hexagonal prisms, etc.), or the like.

The size of each post 58, 58′ may also be characterized by its top surface area, height, and/or diameter.

The top surface area of each post 58, 58′ can be selected based upon the size of the single stranded clustered magnetic bead 10 that is to be anchored to the capture site 46, 46′ that is supported by the post 58, 58′. For example, the top surface area of each post 58, 58′ can be at least about 1×10⁻⁴ μm², at least about 1×10⁻³ μm², at least about 0.1 μm², at least about 1 μm², at least about 10 μm², at least about 100 μm², or more. Alternatively or additionally, the top surface area of each post 58, 58′ can be at most about 1×10⁴ μm², at most about 100 μm², at most about 10 μm², at most about 1 μm², at most about 0.1 μm², at most about 1×10⁻² μm², or less. The area occupied by each post top surface can be greater than, less than or between the values specified above.

The height of each post 58, 58′ can depend upon the flow channel 50 dimensions. In an example, the height may be at least about 0.1 μm, at least about 0.5 μm, at least about 1 μm, at least about 10 μm, at least about 100 μm, or more. Alternatively or additionally, the height can be at most about 1×10³ μm, at most about 100 μm, at most about 10 μm, or less. In some examples, the depth is about 0.4 μm. The height of each post 58, 58′ can be greater than, less than or between the values specified above.

In some instances, the diameter or length and width of each post 58, 58′ can be at least about 50 nm, at least about 0.1 μm, at least about 0.5 μm, at least about 1 μm, at least about 10 μm, at least about 100 μm, or more. Alternatively or additionally, the diameter or length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or length and width is about 0.4 μm. The diameter or length and width of each post 58, 58′ can be greater than, less than or between the values specified above.

In the example shown in FIG. 4B, a respective capture site 46, 46′ is positioned on each of the posts 58, 58′. Any example of the magnetic, chemical, or electrostatic capture sites 46, 46′ described herein may be used.

Magnetic capture sites include any example of the magnetic capture agent set forth herein that can be deposited on the top surface of each post 58, 58′. For example, magnetic materials may be deposited using chemical vapor deposition, masking and deposition, or another suitable technique to form the capture sites 46, 46′.

Chemical capture sites include any example of the chemical capture agent set forth herein that can be deposited on or otherwise attached to the top surface of each post 58, 58′. In one example, the chemical capture agent may be deposited, e.g., using microcontact printing, aerosol printing, etc., on each post 58, 58′ to form the capture site 46, 46′. In another example, a mask (e.g., a photoresist) may be used to cover the interstitial regions 68, 68′ and not the posts 58, 58′. The chemical capture agent may then be deposited on the exposed posts 58, 58′, and the mask removed (e.g., via lift-off, dissolution, or another suitable technique). In this example, the chemical capture agent may form a monolayer or thin layer of the chemical capture agent on the post 58, 58′. In still another example, a polymer grafted with capture nucleic acids may be selectively applied to the top surface of each post 58, 58′ to form the chemical captures sites.

Electrostatic captures sites include any example of the electrostatic capture agent set forth herein that can be deposited on the top surface of each post 58, 58′. For example, electrode materials may be deposited using chemical vapor deposition, masking and deposition, or another suitable technique to form the capture sites 46, 46′. When electrostatic capture sites are used, the substrate 48B, 48B′ may include additional circuitry to address the individual capture sites 46, 46′.

In the example shown in FIG. 4C, the substrate 48C, 48C′ includes depressions 60, 60′ separated by interstitial regions 68, 68′; and a capture site 46, 46′ is positioned in each of the depressions 60, 60′.

Each depression 60, 60′ is a three-dimensional structure that extends inward (downward) from an adjacent surface. The depression 60, 60′ is thus a concave region with respect to the interstitial regions 68, 68′ that surround the depressions 60, 60′. Depressions 60, 60′ may be formed in a substrate 48C, 48C′. In the example shown in FIG. 4C, a material is patterned (e.g., via etching, photolithography, imprinting, etc.) the depressions 60, 60′, which are separated by interstitial regions 68, 68′ that extend above and around each depression 60, 60′. This material is another example of the patterned material 56, 56′.

The layout or pattern of the depressions 60, 60′ may be any of the examples set forth herein for the capture sites 46, 46′. The layout or pattern of the depressions 60, 60′ may be characterized with respect to the density of the depressions 60, 60′ (e.g., number of depressions 60, 60′) in a defined area. Any of the densities set forth for the capture sites 46, 46′ may be used for the depressions 60, 60′. The layout or pattern of the depressions 60, 60′ may also be characterized in terms of the average pitch, or the spacing from the center of one depression 60, 60′ to the center of an adjacent depression 60, 60′ (center-to-center spacing) or from the left edge of one depression 60, 60′ to the right edge of an adjacent depression 60, 60′ (edge-to-edge spacing). Any of the average pitches set forth for the capture sites 46, 46′ may be used for the depressions 60, 60′.

While any suitable three-dimensional geometry may be used for the depressions 60, 60′, a geometry with an at least substantially flat bottom surface may be desirable so that the capture site 46, 46′ may be formed thereon. Example depression geometries include a sphere, a cylinder, a cube, polygonal prisms (e.g., rectangular prisms, hexagonal prisms, etc.), or the like.

The size of each depression 60, 60′ may be characterized by its volume, opening area, depth, and/or diameter.

Each depression 60, 60′ can have any volume that is capable of receiving the material of the capture site 46, 46′. For example, the volume can be at least about 1×10⁻³ μm³, at least about 1×10⁻² μm³, at least about 0.1 μm³, at least about 1 μm³, at least about 10 μm³, at least about 100 μm³, or more. Alternatively or additionally, the volume can be at most about 1×10⁴ μm³, at most about 1×10³ μm³, at most about 100 μm³, at most about 10 μm³, at most about 1 μm³, at most about 0.1 μm³, or less.

The area occupied by each depression opening can be selected based on the size of the single stranded clustered magnetic bead 10 that is to be anchored by the capture site 46, 46′. It may be desirable for the single stranded clustered magnetic bead 10 to enter the depression 60, 60′, and thus the area occupied by the depression opening may be bigger than the size of the single stranded clustered magnetic bead 10. For example, the area for each depression opening can be at least about 1×10⁻³ μm², at least about 1×10⁻² μm², at least about 0.1 μm², at least about 1 μm², at least about 10 μm², at least about 100 μm², or more. Alternatively or additionally, the area can be at most about 1×10³ μm², at most about 100 μm², at most about 10 μm², at most about 1 μm², at most about 0.1 μm², at most about 1×10⁻² μm², or less. The area occupied by each depression opening can be greater than, less than or between the values specified above.

The depth of each depression 60, 60′ is large enough to house at least the capture site 46, 46′. In one example, the depression 60, 60′ may be filled with the capture site 46, 46′. In this example, the single stranded clustered magnetic bead 10 becomes anchored to the capture site 46, 46′ but does not enter the depression 60, 60′. In another example, the depression 60, 60′ may be partially filled with the capture site 46, 46′. In this example, the single stranded clustered magnetic bead 10 at least partially enters the depression 60, 60′ and becomes anchored to the capture site 46, 46′ in the depression 60, 60′. In an example, the depth may be at least about 0.1 μm, at least about 0.5 μm, at least about 1 μm, at least about 10 μm, at least about 100 μm, or more. Alternatively or additionally, the depth can be at most about 1×10³ μm, at most about 100 μm, at most about 10 μm, or less. In some examples, the depth is about 0.4 μm. The depth of each depression 60, 60′ can be greater than, less than or between the values specified above.

In some instances, the diameter or length and width of each depression 60, 60′ can be at least about 50 nm, at least about 0.1 μm, at least about 0.5 μm, at least about 1 μm, at least about 10 μm, at least about 100 μm, or more. Alternatively or additionally, the diameter or length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or length and width is about 0.4 μm. The diameter or length and width of each depression 60, 60′ can be greater than, less than or between the values specified above.

In the example shown in FIG. 4C, the capture site 46, 46′ is positioned in each of the depressions 60, 60′.

In one example, the capture sites 46, 46′ include any example of the magnetic capture agent set forth herein that can be deposited on the bottom surface of each depression 60, 60′. For example, magnetic materials may be deposited using chemical vapor deposition, masking and deposition, or another suitable technique to form the capture sites 46, 46′.

In another example, the capture sites 46, 46′ include any example of the chemical capture agent set forth herein that can be deposited on or otherwise attached to the bottom surface of each depression 60, 60′. In one example, the chemical capture agent may be deposited, e.g., using microcontact printing, aerosol printing, etc., on each depression 60, 60′ to form the capture sites 46, 46′. In another example, a mask (e.g., a photoresist) may be used to cover the interstitial regions 68, 68′ and not the depressions 60, 60′. The chemical capture agent may then be deposited in the exposed depression 60, 60′, and the mask removed (e.g., via lift-off, dissolution, or another suitable technique). In this example, the chemical capture agent may form a monolayer or thin layer of the chemical capture agent in the depression 60, 60′. In still another example, a polymer grafted with capture nucleic acids may be selectively applied to the bottom surface of each depression 60, 60′.

In still another example, the capture sites 46, 46′ include any example of the electrostatic capture agent set forth herein that can be deposited on the bottom surface of each depression 60, 60′. For example, electrode materials may be deposited using chemical vapor deposition, masking and deposition, or another suitable technique to form the capture sites 46, 46′. When electrostatic capture sites are used, the substrate 48C, 48C′ may include additional circuitry to address the individual capture sites 46, 46′.

While the example architectures shown in FIG. 4A, FIG. 4B, and FIG. 4C depict the single stranded clustered magnetic beads 10 anchored at the captures sites 46, 46′, it is to be understood that the flow cell 44 does not include the single stranded clustered magnetic beads 10 until they are introduced thereto, e.g., during sequencing.

Another example of the flow cell architecture is shown in FIG. 4D. This example depicts a flow cell 44′ that in integrated with a complementary metal oxide semiconductor (CMOS) chip 70.

In this example flow cell 44′, the substrate 48D is positioned over the CMOS chip 70. The substrate 48D is similar to the substrate 48A, except that it includes a plurality of depressions 60 defined therein and separated by interstitial regions 68. The capture site 46 is positioned on a bottom surface in each of the plurality of depressions 60.

In this example, the substrate 48D may be a passivation layer. With the passivation layer as the substrate 48D, the substrate 48D may provide one level of corrosion protection for an embedded metal layer 78 of the CMOS chip 70 that is closest in proximity to the substrate 48D. In this example, the substrate 48D may include a passivating material that is transparent to the light emissions (e.g., visible light) resulting from reactions at the capture sites 46, and that is at least initially resistant to the fluidic environment and moisture that may be introduced into or present in the flow channel 50. An “at least initially resistant material” acts as an etch barrier to high pH reagents (e.g., pH ranging from 8 to 14) and as a moisture barrier. Examples of suitable passivation materials for the substrate 48D include silicon nitride (Si₃N₄), silicon oxide (SiO₂), tantalum pentoxide (TaO₅), hafnium oxide (HfO₂), boron doped p+ silicon, or the like. The thickness of the substrate 48D ranges from about 100 nm to about 500 nm.

As mentioned, at least a portion of the substrate 48D (i.e., the passivation layer) is in contact with the first embedded metal layer 78 of the CMOS chip 70 and also with an input region 80 of the optical waveguide 76. The contact between the substrate 48D and the first embedded metal layer 78 may be direct contact or may be indirect contact through a shield layer 82.

In the illustrated example, the substrate 48D may be affixed directly to, and thus be in physical contact with, the CMOS chip 70 through one or more securing mechanisms (e.g., adhesive, bond, fasteners, and the like). It is to be understood that the substrate 48D may be removably coupled to the CMOS chip 70.

The CMOS chip 70 includes a plurality of stacked layers 72 including, for example, silicon layer(s), dielectric layer(s), metal-dielectric layer(s), metal layer(s), etc.). The stacked layers 72 make up the device circuitry, which includes detection circuitry.

The CMOS chip 70 includes optical components, such as optical sensor(s) 74 and optical waveguide(s) 76. The optical components are arranged such that each optical sensor 74 at least substantially aligns with, and thus is operatively associated with, a single optical waveguide 76 and a single reaction site (i.e., each location where the capture site 46 is formed) of the flow cell 44′. However, in other examples, a single optical sensor 74 may receive photons through more than one optical waveguide 76 and/or from more than one reaction site. In these other examples, the single optical sensor 74 is operatively associated with more than one optical waveguide 76 and/or more than one reaction site.

As used herein, a single optical sensor 74 may be a light sensor that includes one pixel or more than one pixel. As an example, each optical sensor 74 may have a detection area that is less than about 50 μm². As another example, the detection area may be less than about 10 μm². As still another example, the detection area may be less than about 2 μm². In the latter example, the optical sensor 74 may constitute a single pixel. An average read noise of each pixel the optical sensor 74 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 76 may be a light guide including a cured filter material that i) filters the excitation light 84 (propagating from an exterior of the flow cell 44′ into the flow channel 50), and ii) permits the light emissions (not shown, resulting from reactions at the reaction site(s)) to propagate therethrough toward corresponding optical sensor(s) 74. In an example, the optical waveguide 76 may be, for example, an organic absorption filter. As a specific example, the organic absorption filter may filter excitation light 84 of about 532 nm wavelength and permit light emissions of about 570 nm or more wavelengths. The optical waveguide 76 may be formed by first forming a guide cavity in a dielectric layer 86, and then filling the guide cavity with a suitable filter material.

The optical waveguide 76 may be configured relative to the dielectric material 86 in order to form a light-guiding structure. For example, the optical waveguide 76 may have a refractive index of about 2.0 so that the light emissions are substantially reflected at an interface between the optical waveguide 76 and the surrounding dielectric material 86. In certain examples, the optical waveguide 76 is selected such that the optical density (OD) or absorbance of the excitation light 84 is at least about 4 OD. More specifically, the filter material may be selected and the optical waveguide 76 may be dimensioned to achieve at least 4 OD. In other examples, the optical waveguide 76 may be configured to achieve at least about 5 OD or at least about 6 OD.

The flow cell 44′ also includes a lid 52 that is operatively connected to the substrate 48D to partially define the flow channel 50 between the substrate 48D (and the reaction site(s) therein or thereon) and the lid 52. The lid 52 may be any material that is transparent to the excitation light 84 that is directed toward the reaction site(s). As examples, the lid 52 may include glass (e.g., borosilicate, fused silica, etc.), plastic, etc. A commercially available example of a suitable borosilicate glass is D 263®, available from Schott North America Inc. Commercially available examples of suitable plastic materials, namely cyclo olefin polymers, are the ZEONOR® products available from Zeon Chemicals L.P.

The lid 52 may be physically connected to the substrate 48D through material 62. The material 62 is/are coupled to a portion of the surface of the substrate 48D, and extends between that surface and an interior surface of the lid 52. In some examples, the material 62 and the lid 52 may be integrally formed such that they 62, 52 are a continuous piece of material (e.g., glass or plastic). In other examples, the material 62 and the lid 52 may be separate components that are coupled to each other. In these other examples, the material 62 may be the same material as, or a different material than the lid 52. As examples, the material 62 may include a curable adhesive layer that bonds the lid 52 to the substrate 48D (at a portion of its surface).

In an example, the lid 52 may be a substantially rectangular block having an at least substantially planar exterior surface 88 and an at least substantially planar interior surface 90 that defines a portion of the flow channel 50. The block may be mounted onto the material 62. Alternatively, the block may be etched to define the interior surface 90 and sidewall(s) that are mounted onto the material 62. For example, a recess may be etched into the transparent block. When the etched block is mounted to the substrate 48D, the recess may become at least part of the flow channel 50.

The lid 52 may include inlet and outlet ports 92, 94 that are configured to fluidically engage other ports (not shown) for directing fluid(s) into the flow channel 50 (e.g., from a reagent cartridge or other fluid storage system component) and out of the flow channel 50 (e.g., to a waste removal system).

The flow channel 50 may be sized and shaped to direct a fluid along the reaction site(s). The height of the flow channel 50 and other dimensions of the flow channel 50 may be configured to maintain a substantially even flow of the fluid along the reaction site(s). The dimensions of the flow channel 50 may also be configured to control bubble formation. In an example, the height of the flow channel 50 may range from about 50 μm to about 400 μm. In another example, the height of the flow channel 50 may range from about 80 μm to about 200 μm. It is to be understood that the height of the flow channel 50 may vary, and may be the greatest when the reaction site is located in a reaction chamber (e.g., depression 60) that is defined in the surface of the substrate 48D. In these examples, the depression 60 increases the height of the flow channel 50 at this particular area.

Each reaction site is a localized region in the substrate 48D where a capture site 46 is positioned and where designated reactions involving the single stranded clustered magnetic bead 10 may occur. In the example shown, each reaction site is a depression 60 having the capture site 46 therein.

In an example, the reaction site is at least substantially aligned with the input region 80 of a single optical waveguide 76. As such, light emissions at the reaction site may be directed into the input region 80, through the waveguide 76, and to an associated optical sensor 74. In other examples, one reaction site 102 may be aligned with several input regions 110 of several optical waveguides 100. In still other examples, several reaction sites 102 may be aligned with one input region 110 of one optical waveguide 76.

The embedded metal layer 78 may be any suitable CMOS metal, such as aluminum (Al), aluminum chloride (AlCu), tungsten (W), nickel (Ni), or copper (Cu). The embedded metal layer 78 is a functioning part of the CMOS AVdd line, and through the stacked layers 72, is also electrically connected to the optical sensor 74. Thus, the embedded metal layer 78 participates in the detection/sensing operation.

It is to be understood that the other optical sensors 74 and associated components may be configured in an identical or similar manner. It is also to be understood, however, that the CMOS chip 70 may not be manufactured identically or uniformly throughout. Instead, one or more optical sensor 74 and/or associated components may be manufactured differently or have different relationships with respect to one another

The stacked layer 72 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 70.

The CMOS chip 70 may be manufactured using integrated circuit manufacturing processes. The CMOS chip 70 may include multiple layers, such as a sensor base/layer 56 (e.g., a silicon layer or wafer). The sensor base may include the optical sensor 74. When the CMOS chip 70 is fully formed, the optical sensor 74 may be electrically coupled to the rest of the circuitry in the stacked layers 72 through gate(s), transistor(s), etc.

As used in reference to FIG. 4D, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked 72 96 also includes a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 86 (e.g., SiO₂). Various metallic elements M1-M5 and dielectric materials 86 may be used, such as those suitable for integrated circuit manufacturing.

In the example shown in FIG. 4D, each of the plurality of metal-dielectric layers L1-L6 includes both metallic elements M1, M2, M3, M4, M5 and dielectric material 86. In each of the layers L1-L6, the metallic elements M1, M2, M3, M4, M5 are interconnected and are embedded within dielectric material 86. In some of the metal-dielectric layers L1-L6, additional metallic elements may also be included. Some of these additional metallic elements may be used to address individual pixels through a row and column selector. The voltages at these elements may vary and switch between about −1.4 V and about 4.4 V depending upon which pixel the device is reading out.

The configuration of the metallic elements M1, M2, M3, M4, M5 and dielectric layer 86 in FIG. 4D is illustrative of the circuitry, and it is to be understood that other examples may include fewer or additional layers and/or may have different configurations of the metallic elements M1-M5.

In the example shown in FIG. 4D, the shield layer 82 is in contact with at least a portion of the substrate 48D. The shield layer 82 has an aperture at least partially adjacent to the input region 80 of the optical waveguide 76. This aperture enables the reaction site (and at least some of the light emissions therefrom) to be optically connected to the waveguide 76. It is to be understood that the shield layer 82 may have an aperture at least partially adjacent to the input region 80 of each optical waveguide 76. The shield layer 82 may extend continuously between adjacent apertures.

The shield layer 82 may include any material that can block, reflect, and/or significantly attenuate the light signals that are propagating through the flow channel 50. The light signals may be the excitation light 84 and/or the light emissions from the reaction site(s). As an example, the shield layer 82 may be tungsten (W).

It is to be understood that the flow cell 44′ may also be used for optical detection.

### Enrichment Kits

The beads 16, 26 and a primer solution containing the 5′-tagged second primers 34 may be included in an enrichment kit. An example of the kit include magnetic beads 16 functionalized with a first oligonucleotide primer 18 of a primer set; a primer solution including a liquid carrier and a 5′-tagged second oligonucleotide primer 34 of the primer set, wherein a 5′-tag 40 of the 5′-tagged second oligonucleotide primer 34 is a first member of a binding pair; and non-magnetic beads 60 including a coating 28 of a second member of the binding pair and having a diameter that is at least ten times larger than each of the plurality of magnetic beads 16.

Any example of the functionalized magnetic beads 16 and the coated non-magnetic beads 26 may be used in the enrichment kit. In one example of the enrichment kit, each the plurality of magnetic beads 16 has a diameter ranging from about 100 nm to about 1000 nm; and the diameter of each of the plurality of coated non-magnetic beads 26 ranges from about 100 nm to about 10 μm.

The functionalized magnetic beads 16 may be suspended in a suitable liquid carrier, or may be lyophilized and resuspended in the liquid carrier before use. Some examples of the enrichment kit also include a flow cell 44, 44′ that includes a plurality of depressions 60, 60′, each of which is configured to receive one of the plurality of magnetic beads 16 (e.g., after clustering is performed off board the flow cell 44, 44′).

### Method for Using the Single Stranded Clustered Magnetic Beads

After the single stranded clustered magnetic beads 10 are formed, they can be introduced into a flow cell 44, 44′ as described herein. At least some of the single stranded clustered magnetic beads 10 will respectively and magnetically attach to at least some of the capture sites 46, 46′. The suspension containing the beads 10 may be allowed to incubate for a predetermined time to allow the single stranded clustered magnetic beads 10 to become anchored. When electrostatic capture sites 22A, 22B, 22C are used, the individual sites 22A, 22B, 22C may be electrically addressed to move the functionalized plasmonic nanostructures 10, 10′ toward individual capture sites 22A, 22B, 22C. In this example, the functionalized plasmonic nanostructures 10, 10′ may include a reversibly chargeable functional group that can be converted from a neutral species to a charged species at a suitable pH. The charged species can be generated by adjusting the pH, and then attracted to the electrostatic capture sites 22A, 22B, 22C that are individually or globally addressed.

A wash cycle may be performed to remove any unanchored single stranded clustered magnetic beads 10.

Sequencing primers may then be introduced to the flow cell 44, 44′. The sequencing primers hybridize to a complementary portion of the sequence of the amplicons 22 that are attached to the single stranded clustered magnetic beads 10. These sequencing primers render the amplicons 22 ready for sequencing.

An incorporation mix including labeled nucleotides may then be introduced into the flow cell 44, 44′, e.g., via the input port 92. In addition to the labeled nucleotides, the incorporation mix may include water, a buffer, and polymerases capable of nucleotide incorporation. When the incorporation mix is introduced into the flow cell 44, 44′, the mix enters the flow channel 40, and contacts the anchored single stranded clustered magnetic beads 10.

The incorporation mix is allowed to incubate in the flow cell 44, 44′, and labeled nucleotides (including optical labels) are incorporated by respective polymerases into the nascent strands along the amplicons 22 on each of the single stranded clustered magnetic beads 10. During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the amplicon 22. Incorporation occurs in at least some of the amplicons 22 across the single stranded clustered magnetic beads 10 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 44, 44′ during a wash cycle. The wash cycle may involve a flow-through technique, where a washing solution (e.g., buffer) is directed into, then through, and then out of flow channel 50, e.g., by a pump or other suitable mechanism.

Without further incorporation taking place, the most recently incorporated labeled nucleotides can be detected through an imaging event or an electronic signal generated in response to an imaging event. During the imaging event, an illumination system may provide an excitation light 84 to the flow cell 44, 44′. The optical labels of the incorporated labeled nucleotides emit optical signals in response to the excitation light. These signals can be detected optically or electronically (e.g., using the flow cell 44′).

After imaging and detection is performed, a cleavage mix may then be introduced into the flow cell 44, 44′. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the amplicons 22 are sequenced.

To further illustrate the present disclosure, an example is given herein. It is to be understood that this example is provided for illustrative purposes and is not to be construed as limiting the scope of the present disclosure.

### Non-Limiting Working Example

250 nm magnetic beads were coated with PAZAM and P7 primers were grafted to the PAZAM. The functionalized magnetic beads were suspended at 0.1 mg/mL in a high salt buffer to form a suspension.

DNA library templates were prepared. The library templates were diluted from 10 nM to 100 pM in a low ionic strength salt buffer and was maintained at 95° C. for about 5 minutes with shaking at 1500 rpm in a thermomixer. After about 5 minutes, the library templates were refrigerated at 4° C. until they were mixed into the suspension containing the functionalized magnetic beads at a ratio of 4:1 bead:library template. The suspension was vortexed and kept in a thermomixer at 40° C. for about 30 minutes with shaking at 1150 rpm. Library templates were seeded to the functionalized magnetic beads during this process. The seeded functionalized magnetic beads were washed with a buffer solution.

First strand extension was then performed by suspending the seeded functionalized magnetic beads in an enzyme mixture (including natural nucleotides, a polymerase, and buffers) and introducing this suspension in the thermomixer at 60° C. for about 10 minutes with shaking at 1150 rpm.

The strand extended functionalized magnetic beads were divided into 4 samples.

A PEG-free clustering mix (containing a modified polymerase) was added to 2 of the samples containing the strand extended functionalized magnetic beads (referred to herein as example samples). Biotinylated 10-mer truncated P5′ primers were added to the example samples. These primers had the complementary sequence of P5, with 10 nucleotides removed from the 5′ end and replaced with biotin. The example samples were respectively maintained at 38° C. for either 30 minutes or 60 minutes on the thermomixer with shaking at 1150 rpm. This allowed strand invasion amplification to take place.

For comparison, the PEG-free clustering mix was added to 2 of the other samples containing the strand extended functionalized magnetic beads (referred to herein as comparative example samples). P5′ primers were added to the comparative example samples. These primers had the complementary sequence of P5, with no biotin added. The comparative example samples were respectively maintained at 38° C. for either 30 minutes or 60 minutes on the thermomixer with shaking at 1150 rpm. This allowed strand invasion amplification to take place.

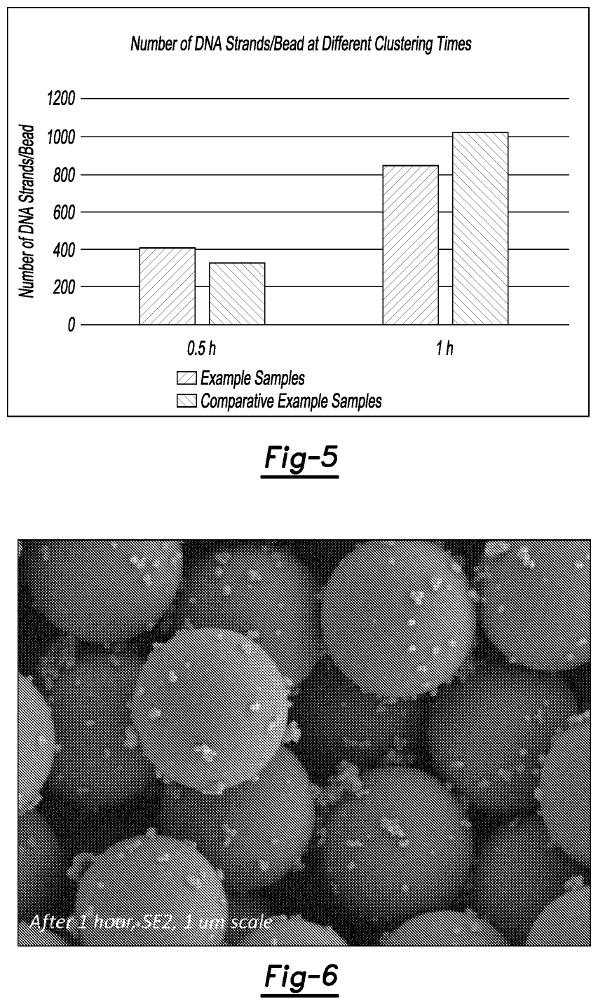

The DNA content on the magnetic beads in each of the example and comparative example samples was determined using qPCR (quantitative polymerase chain reaction), and the results are shown in FIG. 5. As depicted, the clustering efficiency was similar for the example and comparative example samples, illustrating that the biotinylated 10-mer truncated P5′ primers are effective for clustering the library templates on the magnetic bead surface.

Three additional example samples were prepared as described in this example.

One of the additional example samples was used as a control. The DNA content on the magnetic beads of the control sample was determined using qPCR (quantitative polymerase chain reaction), and the results are shown in FIG. 7.

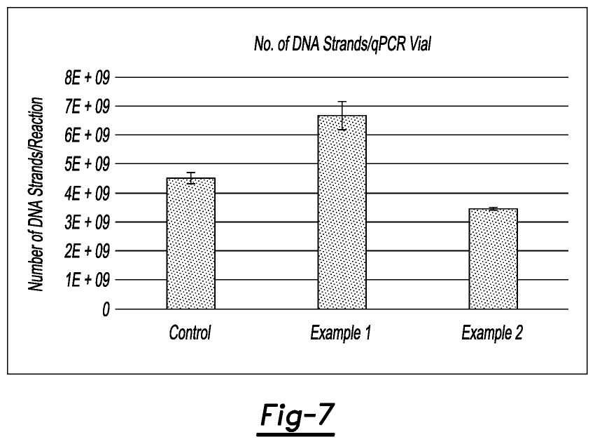

To the other two additional example samples (of clustered magnetic beads), streptavidin coated silica beads (5 μm) were respectively added at 0.1 mg/mL and were allowed to incubate at room temperature (about 18° C.) for about 16 hours with on the thermomixer with shaking at 1150 rpm. After one hour of incubation, a sampling of one of the additional example samples was collected and imaged with scanning electron microscopy (SEM). The SEM image is shown in FIG. 6. As depicted, the biotinylated clustered magnetic beads had begun to attach to the streptavidin coated silica beads even after only one hour of incubation.

The bead-on-bead complexes from the two additional example samples were washed with a buffer solution and respectively exposed to a 0.1 M NaOH solution to dehybridize the double stranded amplicons of the clustered magnetic beads and to dissolve some of the streptavidin coated silica beads. The bead-on-bead complexes in one of the additional example samples (Example 1) were allowed to incubate in the 0.1 M NaOH solution for about 5 minutes at room temperature and the bead-on-bead complexes in the other of the additional example samples (Example 2) were allowed to incubate in the 0.1 M NaOH solution for about 5 minutes at 37° C.

The DNA content on the magnetic beads of each of Examples 1 and 2 was determined using qPCR (quantitative polymerase chain reaction), and the results are shown in FIG. 7. As shown in FIG. 7, the single stranded DNA remained intact on the magnetic beads after dehybridization.

### Clauses

Clause 1. An enrichment method, comprising:
generating a mixture of clustered magnetic beads and unclustered magnetic beads from a plurality of magnetic beads i) functionalized with a first primer of a primer set and ii) contained in a suspension, each of the clustered magnetic beads including a first amplicon attached to the first primer and a 5′-tagged second amplicon hybridized to the first amplicon, wherein a 5′-tag of the 5′-tagged second amplicon is a first member of a binding pair;
introducing a plurality of coated non-magnetic beads to the suspension, each of the plurality of coated non-magnetic beads including a coating of a second member of the binding pair and having a diameter that is at least ten times larger than each of the plurality of magnetic beads, whereby the clustered magnetic beads bind to at least some of the plurality of coated non-magnetic beads to form bead-on-bead complexes and the unclustered magnetic beads remain free in the suspension;
separating the unclustered magnetic beads from the suspension containing the bead-on-bead complexes;
dehybridizing the 5′-tagged second amplicon from the first amplicon, thereby generating single stranded clustered magnetic beads; and
separating the single stranded clustered magnetic beads from the suspension.

Clause 2. The enrichment method as defined in clause 1, wherein generating the mixture of clustered magnetic beads and unclustered magnetic beads from the plurality of magnetic beads in the suspension involves:
introducing a plurality of library templates to the suspension at a magnetic bead:library template ratio of at least 4:1, whereby some of the plurality of library templates respectively hybridize to the first primer of some of the plurality of magnetic beads and some other of the plurality of magnetic beads remain unseeded;
initiating a first primer extension reaction to generate the first amplicons; and initiating strand invasion amplification on the some of the plurality of magnetic beads by introducing a 5′-tagged second primer of the primer set to the plurality of magnetic beads.

Clause 3. The enrichment method as defined in any of clauses 1 or 2, wherein the plurality of coated non-magnetic beads is added to the suspension at a magnetic bead:coated non-magnetic bead ratio ranging from greater than 1:1 to 300:1.

Clause 4. The enrichment method as defined in any of clauses 1 through 3, wherein dehybridizing the 5′-tagged second amplicon from the first amplicon involves introducing, to the suspension, a basic solution that denatures the 5′-tagged second amplicon from the first amplicon and that dissolves at least a portion of the plurality of coated non-magnetic beads.

Clause 5. The enrichment method as defined in clause 4, wherein the basic solution is an aqueous solution of sodium hydroxide.

Clause 6. The enrichment method as defined in clause 4, wherein the suspension and the basic solution are incubated at a temperature ranging from about 18° C. to about 22° C. for a time ranging from about 3 minutes to about 10 minutes.

Clause 7. The enrichment method as defined in any of clauses 1 through 3, wherein dehybridizing the 5′-tagged second amplicon from the first amplicon involves introducing, to the suspension, formamide.

Clause 8. The enrichment method as defined in clause 7, wherein the suspension and the formamide are incubated at a temperature ranging from about 55° C. to about 65° C. for a time ranging from about 15 minutes to about 30 minutes.

Clause 9. The enrichment method as defined in any one of clauses 1 through 8, wherein the plurality of coated non-magnetic beads are introduced into the suspension in a buffer containing from about 0.1% active (w/v) to about 0.5% active (w/v) of a non-ionic surfactant.

Clause 10. The enrichment method as defined in any one of clauses 1 through 9, further comprising incubating the plurality of coated non-magnetic beads and the suspension for up to 24 hours before the unclustered magnetic beads are filtered from the suspension containing the bead-on-bead complexes.

Clause 11. The enrichment method as defined in any one of clauses 1 through 10, wherein the coated non-magnetic beads are coated silica beads.

Clause 12. The enrichment method as defined in any one of clauses 1 through 11, further comprising introducing the single stranded clustered magnetic beads into a flow cell including a plurality of depressions, each of which is configured to receive one of the single stranded clustered magnetic beads.

Clause 13. The enrichment method as defined in any one of clauses 1 through 12, wherein:
the first member of the binding pair is biotin and the second member of the binding pair is streptavidin; or
the first member of the binding pair is NiNTA (nickel-nitrilotriacetic acid) ligand and the second member of the binding pair is a histidine tag; or
the first and second members of the binding pair are complementary DNA strands that can hybridize to one another; or
the first and second members of the binding pair are functional groups that can form a disulfide bond; or
the first and second members of the binding pair are functional groups that can form an imine.

Clause 14. An enrichment kit, comprising:
magnetic beads functionalized with a first oligonucleotide primer of a primer set;
a primer solution including a liquid carrier and a 5′-tagged second oligonucleotide primer of the primer set, wherein a 5′-tag of the 5′-tagged second oligonucleotide primer is a first member of a binding pair; and
non-magnetic beads including a coating of a second member of the binding pair and having a diameter that is at least ten times larger than each of the plurality of magnetic beads.

Clause 15. The enrichment kit as defined in clause 14, wherein:
each of the plurality of magnetic beads has a diameter ranging from about 100 nm to about 1000 nm; and
the diameter of each of the plurality of coated non-magnetic beads ranges from about 100 nm to about 10 μm.

Clause 16. The enrichment kit as defined in any of clauses 14 or 15, further comprising a flow cell including a plurality of depressions, each of which is configured to receive one of the plurality of magnetic beads.

Clause 17. The enrichment kit as defined in any of clauses 14 through 16, wherein:
a surface of each of the magnetic beads has a polymeric hydrogel coating applied thereon; and
the first oligonucleotide primer is attached to the polymeric hydrogel.

Clause 18. The enrichment kit as defined in any of clauses 14 through 17, wherein:
the first member of the binding pair is biotin and the second member of the binding pair is streptavidin; or
the first member of the binding pair is NiNTA (nickel-nitrilotriacetic acid) ligand and the second member of the binding pair is a histidine tag; or
the first and second members of the binding pair are complementary DNA strands that can hybridize to one another; or
the first and second members of the binding pair are functional groups that can form a disulfide bond; or
the first and second members of the binding pair are functional groups that can form an imine.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range from about 2 mm to about 300 mm, should be interpreted to include not only the explicitly recited limits of from about 2 mm to about 300 mm, but also to include individual values, such as about 40 mm, about 250.5 mm, etc., and sub-ranges, such as from about 25 mm to about 175 mm, etc.

Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

What is claimed is:

1. An enrichment method, comprising:
generating a mixture of clustered magnetic beads and unclustered magnetic beads from a plurality of magnetic beads i) functionalized with a first primer of a primer set and ii) contained in a suspension, each of the clustered magnetic beads including a first amplicon attached to the first primer and a 5′-tagged second amplicon hybridized to the first amplicon, wherein a 5′-tag of the 5′-tagged second amplicon is a first member of a binding pair;
introducing a plurality of coated non-magnetic beads to the suspension, each of the plurality of coated non-magnetic beads including a coating of a second member of the binding pair and having a diameter that is at least ten times larger than each of the plurality of magnetic beads, whereby the clustered magnetic beads bind to at least some of the plurality of coated non-magnetic beads to form bead-on-bead complexes and the unclustered magnetic beads remain free in the suspension;
separating the unclustered magnetic beads from the suspension containing the bead-on-bead complexes;
dehybridizing the 5-tagged second amplicon from the first amplicon, thereby generating single stranded clustered magnetic beads; and
separating the single stranded clustered magnetic beads from the suspension.

2. The enrichment method as defined in claim 1, wherein generating the mixture of clustered magnetic beads and unclustered magnetic beads from the plurality of magnetic beads in the suspension involves:
introducing a plurality of library templates to the suspension at a magnetic bead:library template ratio of at least 4:1, whereby some of the plurality of library templates respectively hybridize to the first primer of some of the plurality of magnetic beads and some other of the plurality of magnetic beads remain unseeded;
initiating a first primer extension reaction to generate the first amplicons; and
initiating strand invasion amplification on the some of the plurality of magnetic beads by introducing a 5′-tagged second primer of the primer set to the plurality of magnetic beads.

3. The enrichment method as defined in claim 1, wherein the plurality of coated non-magnetic beads is added to the suspension at a magnetic bead:coated non-magnetic bead ratio ranging from greater than 1:1 to 300:1.

4. The enrichment method as defined in claim 1, wherein dehybridizing the 5′-tagged second amplicon from the first amplicon involves introducing, to the suspension, a basic solution that denatures the 5′-tagged second amplicon from the first amplicon and that dissolves at least a portion of the plurality of coated non-magnetic beads.

5. The enrichment method as defined in claim 4, wherein the basic solution is an aqueous solution of sodium hydroxide.

6. The enrichment method as defined in claim 4, wherein the suspension and the basic solution are incubated at a temperature ranging from about 18° C. to about 22° C. for a time ranging from about 3 minutes to about 10 minutes.

7. The enrichment method as defined in claim 1, wherein dehybridizing the 5′-tagged second amplicon from the first amplicon involves introducing, to the suspension, formamide.

8. The enrichment method as defined in claim 7, wherein the suspension and the formamide are incubated at a temperature ranging from about 55° C. to about 65° C. for a time ranging from about 15 minutes to about 30 minutes.

9. The enrichment method as defined in claim 1, wherein the plurality of coated non-magnetic beads are introduced into the suspension in a buffer containing from about 0.1% active (w/v) to about 0.5% active (w/v) of a non-ionic surfactant.

10. The enrichment method as defined in claim 1, further comprising incubating the plurality of coated non-magnetic beads and the suspension for up to 24 hours before the unclustered magnetic beads are filtered from the suspension containing the bead-on-bead complexes.

11. The enrichment method as defined in claim 1, wherein the coated non-magnetic beads are coated silica beads.

12. The enrichment method as defined in claim 1, further comprising introducing the single stranded clustered magnetic beads into a flow cell including a plurality of depressions, each of which is configured to receive one of the single stranded clustered magnetic beads.

13. The enrichment method as defined in claim 1, wherein:
the first member of the binding pair is biotin and the second member of the binding pair is streptavidin; or
the first member of the binding pair is NiNTA (nickel-nitrilotriacetic acid) ligand and the second member of the binding pair is a histidine tag; or
the first and second members of the binding pair are complementary DNA strands that can hybridize to one another; or
the first and second members of the binding pair are functional groups that can form a disulfide bond; or
the first and second members of the binding pair are functional groups that can form an imine.

14. An enrichment kit, comprising:
magnetic beads functionalized with a first oligonucleotide primer of a primer set;
a primer solution including a liquid carrier and a 5′-tagged second oligonucleotide primer of the primer set, wherein a 5′-tag of the 5′-tagged second oligonucleotide primer is a first member of a binding pair; and
non-magnetic beads including a coating of a second member of the binding pair and having a diameter that is at least ten times larger than each of the plurality of magnetic beads.

15. The enrichment kit as defined in claim 14, wherein:
each of the plurality of magnetic beads has a diameter ranging from about 100 nm to about 1000 nm; and
the diameter of each of the plurality of coated non-magnetic beads ranges from about 100 nm to about 10 μm.

16. The enrichment kit as defined in claim 14, further comprising a flow cell including a plurality of depressions, each of which is configured to receive one of the plurality of magnetic beads.

17. The enrichment kit as defined in claim 14, wherein:
a surface of each of the magnetic beads has a polymeric hydrogel coating applied thereon; and
the first oligonucleotide primer is attached to the polymeric hydrogel.

18. The enrichment kit as defined in claim 14, wherein:
the first member of the binding pair is biotin and the second member of the binding pair is streptavidin; or
the first member of the binding pair is NiNTA (nickel-nitrilotriacetic acid) ligand and the second member of the binding pair is a histidine tag; or
the first and second members of the binding pair are complementary DNA strands that can hybridize to one another; or
the first and second members of the binding pair are functional groups that can form a disulfide bond; or
the first and second members of the binding pair are functional groups that can form an imine.


SEQUENCE LISTING (7 sequences)

SEQ ID NO: 1 - DNA, 28 bp, synthetic construct
  source 1..28: mol_type = other DNA
aatgatacggcgaccaccgagactacac

SEQ ID NO: 2 - DNA, 23 bp, synthetic construct
  source 1..23: mol_type = other DNA
caagcagaagacggcatacgaat

SEQ ID NO: 3 - DNA, 35 bp, synthetic construct
  source 1..35: mol_type = other DNA
gctggcacgtccgaacgcttcgttaatccgttgag

SEQ ID NO: 4 - DNA, 35 bp, synthetic construct
  source 1..35: mol_type = other DNA
cgtcgtctgccatggcgcttcggtggatatgaact

SEQ ID NO: 5 - DNA, 35 bp, synthetic construct
  source 1..35: mol_type = other DNA
acggccgctaatatcaacgcgtcgaatccgcaact

SEQ ID NO: 6 - DNA, 34 bp, synthetic construct
  source 1..34: mol_type = other DNA
gccgcgttacgttagccggactattcgatgcagc

SEQ ID NO: 7 - DNA, 13 bp, synthetic construct
  source 1..13: mol_type = other DNA
tacggcgaccacc